(19) United States
(12) Patent Application Publication
(10) Pub. No.: US 2005/0074847 A1
(43) Pub. Date: Apr. 7, 2005

(54) Human prostaglandin EP1 receptor variants and methods of using same

(76) Inventors: Yanbin Liang, Irvine, CA (US); David F. Woodward, Lake Forest, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 10/681,029

(22) Filed: Oct. 7, 2003

(51) Int. Cl. C07K 14/705; C07H 21/04

(52) U.S. Cl. 435/69.1; 435/320.1; 435/325; 530/350; 536/23.5

(57) ABSTRACT

The present invention relates to nucleic acid molecules encoding novel, alternatively spliced EP₁ receptor variants. Variant EP₁ polypeptides and screening methods based on such polypeptides also are provided.

### BACKGROUND OF THE INVENTION

This invention relates generally to molecular medicine and, more specifically, to alternatively spliced prostaglandin EP₁ receptors.

Prostaglandins (PG) and thromboxane, collectively named prostanoids, are oxygenated fatty acids that bind to seven transmembrane domain G-protein coupled receptors (GPCRs). The classification of prostanoid receptors into DP, EP, FP, IP, and TP is based on the binding and functional potency of the five naturally occurring prostanoids, PGD₂′ PGE₂′ PGF_{2α}I₂, and TXA₂, respectively. Prostanoid receptors have been cloned and expressed in cultured cells, where ligand binding and signal transduction properties have been studied. It is recognized that prostanoids can bind to more than one prostanoid receptor type; however, each prostanoid binds to its respective receptor with an affinity at least one order of magnitude higher than its affinity for the other four prostanoid receptors.

Prostanoids produce numerous physiologic and pathophysiologic effects and regulate cellular processes in nearly every tissue. The wide spectrum of prostanoid action includes effects on immune, endocrine, cardiovascular, renal and reproductive systems as well as the contraction and relaxation of smooth muscle. Accordingly, prostanoids and prostanoid analogues have been used as drugs to treat a variety of clinical conditions, including, but not limited to, various types of pain.

Although the broad classification of prostanoid receptors into five classes remains intact, the differential effects of PGE₂ on target tissues provides evidence for a subdivision within the EP receptor family. To date, four subtypes of EP receptors, termed EP₁, EP₂, EP₃, and EP₄ have been cloned and expressed. EP₁ receptors are expressed in a range of different species and in a several tissues, including, without limitation, trachea, the gastrointestinal tract, uterus, kidney, lung, gastric fundus, iris sphincter, myometrium and bladder, where they mediate smooth muscle contraction. Recently, a role for the EP₁ receptor in pain has been confirmed by genetic studies using a mouse targeted gene disruption model.

Although some EP receptor subtype selective ligands exist, many of these compounds act at multiple PG receptor subtypes. For example, the EP₁ selective agonists sulprostone also acts at the EP₃ receptor, and the EP₁ selective agonist iloprost also acts at the IP receptor. Thus, while compounds have been synthesized that have reduced agonist activity at other prostanoid receptors, the compounds currently available still have some agonist activity at other receptors, which can result in undesirable side effects. Such side effects may be due in part to a lack of receptor specificity.

A goal of clinical pharmacology and the pharmaceutical industry is the development of more selective drugs with greater efficacy and fewer side effects than those currently in use. In order to more effectively treat conditions such as pain where EP₁ receptor modulators can be of benefit new receptors related to the known wild-type EP₁ receptor must be discovered and used to design screening assays for identification of compounds that bind more specifically to the known EP₁ receptor. Newly identified EP₁ receptors such as alternatively spliced EP₁ receptors can be more closely associated with a condition such as pain than the known EP₁ receptor and can be targets for drug discovery efforts, resulting in the development of drugs having greater efficacy or fewer side effects than drugs developed against the known wild-type EP₁ receptor.

Thus, there exists a need for the discovery of new EP₁ receptors which can be used, for example, to design more specific drugs with fewer side effects. The present invention satisfies this need and provides related advantages as well.

### SUMMARY OF THE INVENTION

The present invention provides an isolated polypeptide containing an amino acid sequence having at least 50% amino acid identity with SEQ ID NO: 8, and containing the amino acid sequence of SEQ ID NO: 4 or 6, or a conservative variant of SEQ ID NO: 4 or SEQ ID NO: 6. An isolated polypeptide of the invention can have, for example, at least 80% amino acid identity with SEQ ID NO: 8, or at least 90% amino acid identity with SEQ ID NO: 8 in addition to containing the amino acid sequence of SEQ ID NO: 4 or 6, or a conservative variant thereof. In one embodiment, the invention provides an isolated polypeptide containing the amino acid sequence of SEQ ID NO: 2, or a conservative variant thereof. In further embodiments, an isolated polypeptide of the invention contains the amino acid sequence of SEQ ID NO: 2 or consists of the amino acid sequence of SEQ ID NO: 2. Cells containing an exogenously expressed polypeptide of the invention also are provided.

Further provided by the invention is a method for identifying a compound that modulates an EP₁ receptor variant by contacting an isolated EP₁ receptor variant or an EP₁ receptor variant over-expressed in a genetically engineered cell with a compound, where the EP₁ receptor variant has at least 50% amino acid identity with SEQ ID NO: 8, and contains the amino acid sequence of SEQ ID NO: 4 or 6, or a conservative variant of SEQ ID NO: 4 or SEQ ID NO: 6; and determining the level of an indicator which correlates with modulation of the EP₁ receptor variant, where an alteration in the level of the indicator-as compared to a control level indicates that the compound is a compound that modulates the EP₁ receptor variant. In one embodiment, a screening method of the invention is practiced with an EP₁ receptor variant which is a polypeptide containing an amino acid sequence having at least 80% amino acid identity with SEQ ID NO: 8 and containing the amino acid sequence of SEQ ID NO: 4 or 6, or a conservative variant of SEQ ID NO: 4 or SEQ ID NO: 6. In another embodiment, a screening method of the invention is practiced with an EP₁ receptor variant which is a polypeptide containing the amino acid sequence of SEQ ID NO: 2, or a conservative variant thereof. It is understood that isolated EP₁ receptor variants, as well as EP₁ receptor variants over-expressed in genetically engineered cells, can be useful in the methods of the invention. Furthermore, indicators useful in a method of the invention include, without limitation, calcium, and compounds to be screened according to a method of the invention encompass, but are not limited to, polypeptides and small molecules.

Further provided herein is a method for identifying a compound that specifically binds to an EP₁ receptor variant by contacting an isolated EP₁ receptor variant or an EP₁ receptor variant over-expressed in a genetically engineered cell with a compound, and determining specific binding of the compound to the EP₁ receptor variant, where the EP₁ receptor variant has at least 50% amino acid identity with SEQ ID NO: 8 and contains the amino acid sequence of SEQ ID NO: 4 or 6, or a conservative variant thereof. Isolated EP₁ receptor variants, as well as EP₁ receptor variants over-expressed in genetically engineered cells, can be useful in the screening methods of the invention. In one embodiment, a screening method of the invention is practiced with an EP₁ receptor variant which is a polypeptide containing an amino acid sequence having at least 80% amino acid identity with SEQ ID NO: 8 and containing the amino acid sequence of SEQ ID NO: 4 or 6, or a conservative variant thereof. In a further embodiment, a screening method of the invention is practiced with an EP₁ receptor variant which is a polypeptide containing the amino acid sequence of SEQ ID NO: 2, or a conservative variant thereof. Indicators useful in the methods of the invention include, without limitation, calcium, and compounds to be screened according to the methods of the invention encompass, without limitation, polypeptides and small molecules.

The present invention also provides a method for identifying a compound that differentially modulates an EP₁ receptor variant by contacting an isolated EP₁ receptor variant or an EP₁ receptor variant over-expressed in a genetically engineered cell with a compound, where the EP₁ receptor variant has at least 50% amino acid identity with SEQ ID NO: 8 and contains the amino acid sequence of SEQ ID NO: 4 or 6 or a conservative variant thereof; determining the level of an indicator which correlates with modulation of the EP₁ receptor variant; contacting a second receptor with the compound; determining the level of a corresponding indicator which correlates with modulation of the second receptor; and comparing the level of the indicator with the level of the corresponding indicator, where a different level of the indicator as compared to the level of the corresponding indicator indicates that the compound is a compound that differentially modulates the EP₁ receptor variant. In one embodiment, the second receptor is a different EP₁ receptor variant. In another embodiment, the second receptor includes the the amino acid sequence of SEQ ID NO: 8, or a functional fragment thereof. In a further embodiment, the EP₁ receptor variant is polypeptide containing an amino acid sequence having at least 80% amino acid identity with SEQ ID NO: 8 in addition to containing the amino acid sequence of SEQ ID NO: 4 or 6, or a conservative variant thereof. In yet a further embodiment, the EP₁ receptor variant is a polypeptide containing the amino acid sequence of SEQ ID NO: 2, or a conservative variant thereof. Such variants can be provided as isolated polypeptides or can be over-expressed in genetically engineered cells. Indicators useful in the methods of the invention include, without limitation, calcium. Compounds to be screened according to the methods of the invention include, but are not limited to, polypeptides and small molecules.

Further provided herein is a method for identifying a compound that differentially binds to an EP₁ receptor variant by contacting an isolated EP₁ receptor or an EP₁ receptor variant over-expressed in a genetically engineered cell with a compound, where the EP₁ receptor variant has at least 50% amino acid identity with SEQ ID NO: 8 and contains the amino acid sequence of SEQ ID NO: 4 or 6 or a conservative variant thereof; determining specific binding of the compound to the EP₁ receptor variant; contacting a second receptor with the compound; determining specific binding of the compound to the second receptor; and comparing the level of specific binding to the EP₁ receptor variant with the level of specific binding to the second receptor, where a different level of specific binding to the EP₁ receptor variant as compared to the second receptor indicates that the compound is a compound that differentially binds to the EP₁ receptor variant. A method of the invention for identifying a compound that differentially binds to an EP₁ receptor variant can be practiced, for example, with a second receptor which is a different EP₁ receptor variant, or with a second receptor which includes the amino acid sequence of SEQ ID NO: 8, or a functional fragment thereof. EP₁ receptor variants useful in the invention include isolated polypeptides and polypeptides over-expressed in genetically engineered cells. EP₁ receptor variants useful in the invention further include polypeptides containing an amino acid sequence having at least 80% amino acid identity with SEQ ID NO: 8 and containing the amino acid sequence of SEQ ID NO: 4 or 6 or a conservative variant of SEQ ID NO: 4 or 6; and polypeptides containing the amino acid sequence of SEQ ID NO: 2, or conservative variants thereof. Useful indicators include, without limitation, calcium, while compounds to be screened according to a method of the invention include, but are not limited to, polypeptides and small molecules.

### BRIEF DESCRIPTION OF THE DRAWINGS

FIG. 1 shows the nucleotide sequence of EP₁ receptor variant EP₁ VAR-1 (SEQ ID NO: 1). The underlined sequence indicates novel nucleotide sequence compared to the nucleotide sequence of the known wild-type human EP₁ receptor (SEQ ID NO: 7). The start and stop codons for EP₁ receptor variant EP₁ VAR-1 are indicated in bolded capital letters.

FIG. 2 shows a comparison of the amino acid sequences of the known wild-type human EP₁ receptor (SEQ ID NO: 8), abbreviated as WT EP₁, with human EP₁ receptor variant EP₁ VAR-1 (SEQ ID NO: 2). An arrow in the carboxy terminal area of the polypeptides indicates the location where the wild-type human EP₁ receptor and the EP₁ receptor variant EP₁ VAR-1 begin to differ.

FIG. 3 shows distribution of mRNA from EP₁ receptor variant EP₁ VAR-1 in various tissues using multiple tissue Northern blot analysis. The location of EP₁ VAR-1 mRNA is indicated by an arrow. The size of 18S and 28S ribosomal RNA is indicated in lanes 9 and 10. Ciliary SM=ciliary smooth muscle. The probe used in Northern blot analysis contains the intron sequence (labeled “A” in FIG. 5) which is present in EP₁ VAR-1 mRNA.

FIG. 4 shows a comparison of the hydropathy of human wild-type EP₁ receptor (SEQ ID NO: 8) and EP₁ receptor variant EP₁ VAR-1 (SEQ ID NO: 2).

FIG. 5 shows a comparison of the genomic structure of human wild-type EP₁ receptor and EP, receptor variant EP₁ VAR-1. EP₁ receptor variant EP₁ VAR-1 retains an intron (labeled “A”) that is spliced out of the wild-type EP₁ receptor.

### DETAILED DESCRIPTION OF THE INVENTION

The present invention is directed to the exciting discovery of novel EP₁ receptor variants. EP₁ receptor variants can be used to determine and refine the specificity of binding of compounds that bind to the known wild-type EP₁ receptor. EP₁ receptor variants also can be used to identify compounds that differentially modulate or bind to a first EP₁ receptor variant in relation to a second EP₁ receptor variant or wild-type EP₁ receptor. Such a compound can be, for example, a ligand that specifically binds to a novel EP₁ receptor variant disclosed herein.

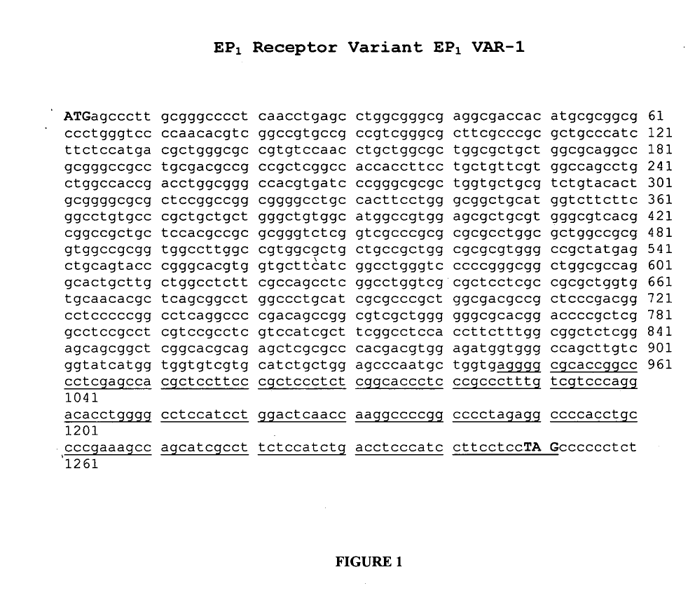

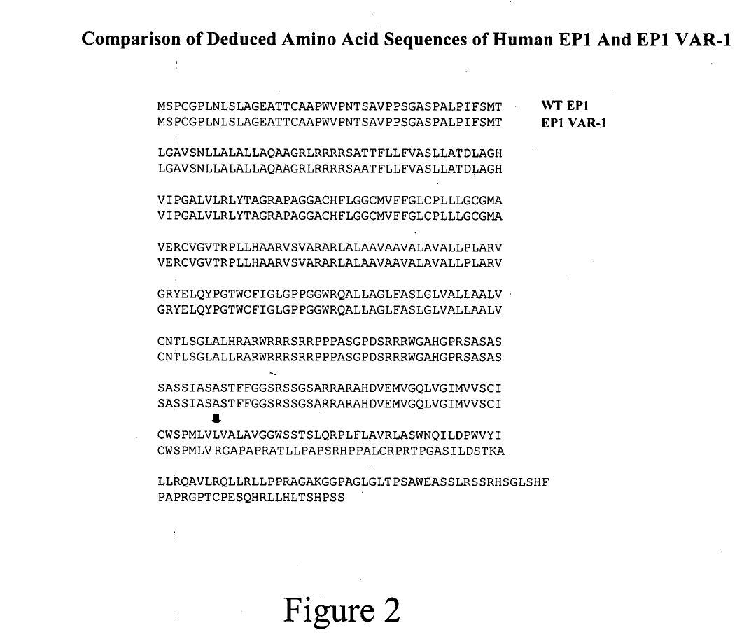

As disclosed herein in Example I, a novel EP₁ receptor variant, EP₁ VAR-1, was identified using the reverse transcription polymerase chain reaction (RT-PCR) and the following EP₁ receptor primers: GGTATCATGGTGGTGTCGTG (SEQ ID NO: 9) and CCTGGCGCAGTAGGATGTA (SEQ ID NO: 10). In particular, the novel alternatively spliced EP₁ receptor variant EP₁ VAR-1 was identified as distinct from the wild-type human EP₁ receptor (see FIGS. 1 and 2).

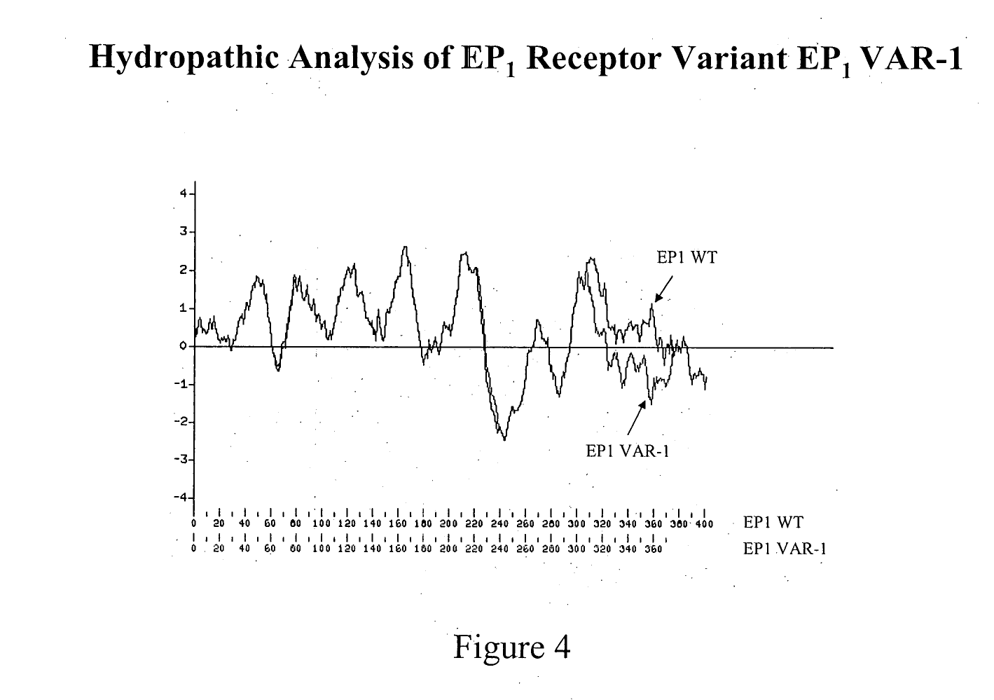

As further disclosed herein, sequence analysis of nucleic acid molecules encoding the alternatively spliced EP₁ receptor variant EP₁ VAR-1 revealed novel carboxy-terminal amino acid sequence. As shown in FIG. 2, the novel carboxy-terminal amino acid sequence in EP₁ receptor variant EP₁ VAR-1 has the sequence RGAPAPRATLLPAPSRHPPALCRPRTPGASILDSTKAPAPRGPTCPESQHRLLHLTS HPSS (SEQ ID NO: 4). Furthermore, the unique carboxy-terminal region of EP₁ receptor variant EP, VAR-1 results in a different hydropathic profile for the EP₁ VAR-1 polypeptide compared to the wild-type EP₁ receptor polypeptide (see FIG. 4).

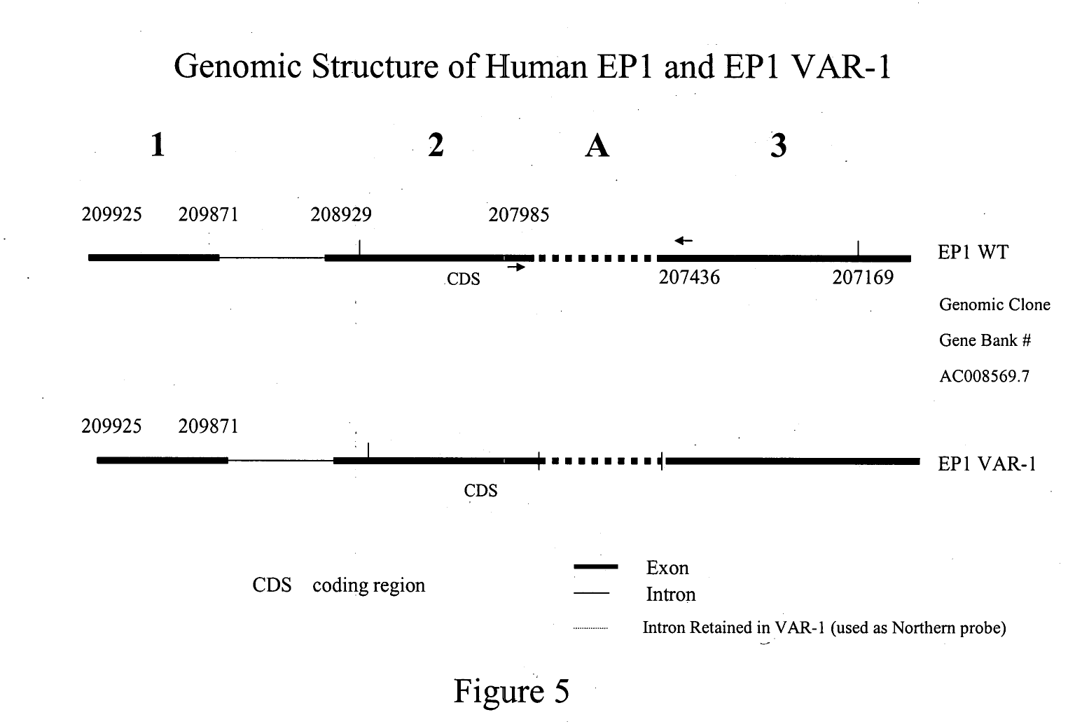

As disclosed herein, EP₁ receptor variant EP₁ VAR-1 retains an intron (labeled “A” in FIG. 5) which is spliced out of the known wild-type human EP₁ receptor. The retained intron in EP₁ receptor variant EP₁ VAR-1 causes a frameshift and premature stop codon which results in different amino acid sequence from the wild-type human EP₁ receptor. As shown in FIGS. 2 and 5, comparison of the known wild-type human EP₁ receptor amino acid sequence (SEQ ID NO: 8) to the alternatively spliced human EP₁ receptor variant EP₁ VAR-1-(SEQ ID NO: 2) revealed the amino acid sequence at the junction between conserved exon 2 and retained intron A within EP₁ receptor variant EP₁ VAR-1 to be PMLVRGAPAP (SEQ ID NO: 6), where the first four amino acids correspond to amino acid sequence present in conserved exon 2 and the remaining six amino acids are residues derived from retained intron A which is present in EP₁ receptor variant EP₁ VAR-1.

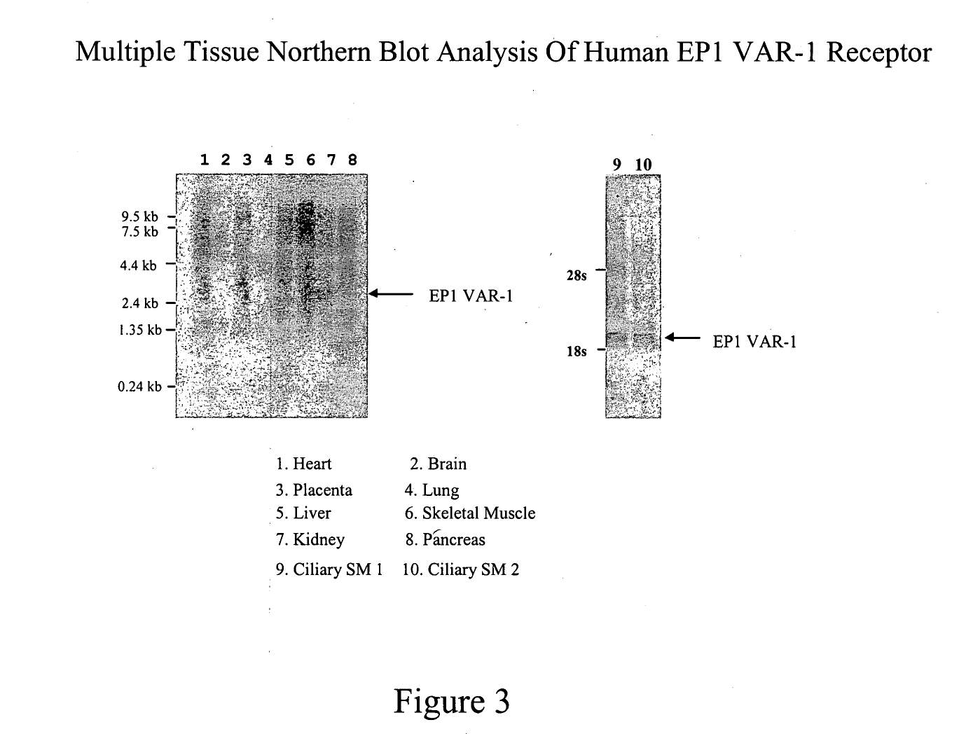

Also disclosed herein is the tissue distribution pattern of EP₁ receptor variant EP₁ VAR-1 (see FIG. 3). Multiple tissue Northern Blot analysis was performed using retained intron A as a probe. As shown in FIG. 3, EP₁ receptor variant EP₁ VAR-1 mRNA was detected at highest levels in kidney and ciliary smooth muscle derived from ocular tissue (also see Example II).

Based on these discoveries, the present invention provides novel alternatively spliced EP₁ receptor variants and screening methods that rely on these variants. In particular, the invention provides an isolated polypeptide containing an amino acid sequence having at least 50% amino acid identity with SEQ ID NO: 8 and containing the amino acid sequence of SEQ ID NO: 4 or 6, or a conservative variant of SEQ ID NO: 4 or 6. Also provided herein is an isolated polypeptide containing the amino acid sequence of SEQ ID NO: 2, or a conservative variant thereof.

The present invention further provides a method for identifying a compound that modulates an EP₁ receptor variant by contacting an isolated EP₁ receptor variant or an EP₁ receptor variant over-expressed in a genetically engineered cell with a compound and determining the level of an indicator which correlates with modulation of an EP₁ receptor variant, where an alteration in the level of the indicator as compared to a control level indicates that the compound is a compound that modulates the EP₁ receptor variant. The present invention also provides a method for identifying a compound that specifically binds to an EP₁ receptor variant by contacting an isolated EP₁ receptor variant or an EP₁ receptor variant over-expressed in a genetically engineered cell with a compound and determining specific binding of the compound to the EP₁ receptor variant.

The invention further provides a method for identifying a compound that differentially modulates an EP₁ receptor variant by a) contacting an isolated EP₁ receptor variant or an EP₁ receptor variant over-expressed in a genetically engineered cell with a compound; b) determining the level of an indicator which correlates with modulation of an EP₁ receptor variant; c) contacting a second receptor with the compound; d) determining the level of a corresponding indicator after contacting of the compound to the second receptor; and e) comparing the level of the indicator from step (b) with the level of the corresponding indicator from step (d), where a different level of the indicator from step (b) compared to the level of the corresponding indicator from step (d) indicates that the compound is a compound that differentially modulates the EP₁ receptor variant.

Further provided herein is a method for identifying a compound that differentially binds to an EP₁ receptor variant by a) contacting an isolated EP₁ receptor variant or an EP₁ receptor variant over-expressed in a genetically engineered cell with a compound; b) determining specific binding of the compound to the EP₁ receptor variant; c) contacting a second receptor with the compound; d) determining specific binding of the compound to the second receptor; and e) comparing the level of specific binding from step (b) with the level of specific binding from step (d), where a different level of specific binding from step (b) compared to the level of specific binding from step (d) indicates that the compound is a compound that differentially binds to the EP₁ receptor variant.

The methods of the invention can be useful for designing drugs that bind to or modulate the wild-type human EP₁ receptor (SEQ ID NO: 8) in preference to one or more alternatively EP₁ receptor variants or for identifying compounds that bind to or modulate one or more EP₁ receptor variants in preference to other EP₁ receptor variants or the wild type EP₁ receptor. Compounds identified by a method of the invention can be therapeutically useful in preventing or reducing the severity of a condition where modulation of an EP₁ receptor or an EP₁ receptor variant is beneficial.

Prostaglandin E₂ (PGE₂) is involved in a number of physiologic and pathophysiologic events in many tissues of the body. The biologic effects of PGE₂ are mediated through interaction with specific membrane-bound G protein-coupled prostanoid EP receptors. Subtypes of the EP receptor, termed EP₁ (PTGER1), EP₂ (PTGER2; 176804), EP₃ (PTGER3), and EP₄ (PTGER4), are defined on the basis of their pharmacologic profiles and signal transduction pathways as reviewed by Coleman et al. Pharm. Rev. 46:205-229 (1994).

From a human erythroleukemia cell cDNA library, Funk et al. isolated a functional cDNA clone coding for the EP₁ receptor (Funk et al., J. Biol. Chem. 268: 26767-26772, (1993)). The EP₁ receptor has 402 amino acids with a predicted molecular mass of 41,858 and the 7 predicted transmembrane-spanning domains common to all G protein-coupled receptors. The EP₁ receptor is functionally coupled to an increase in intracellular calcium ion when expressed in Xenopus oocytes. Duncan et al. mapped the EP₁ receptor to 19p13.1 by in situ hybridization (Duncan et al., Genomics 25:740-742 (1995)).

To identify the physiologic roles of the EP₁ receptor, Stock et al. generated EP₁ receptor −/− mice using homologous recombination in embryonic stem cells (Stock et al., J. Clin. Invest. 107:325-331 (2001)). The EP₁ receptor −/− mice were healthy and fertile, without overt physical defects. However, their pain sensitivity responses, tested in two acute prostaglandin-dependent models, were reduced by approximately 50%. This reduction in the perception of pain was virtually identical to that achieved through pharmacologic inhibition of prostaglandin synthesis in wild-type mice using a cyclooxygenase inhibitor. In addition, systolic blood pressure was significantly reduced in EP₁ receptor-deficient mice and accompanied by increased renin-angiotensin activity, especially in males, implicating a role for this receptor in cardiovascular homeostasis. Thus, the EP₁ receptor for PGE₂ plays a role in mediating algesia and in regulation of blood pressure.

Activation of the hypothalamo-pituitary-adrenal (HPA) axis is one of various neural responses to disease. This response can be induced experimentally by injection of bacterial lipopolysaccharide (LPS) or inflammatory cytokines such as interleukin-1. Matsuoka et al. noted that although prostaglandins had long been implicated in LPS-induced HPA axis activation, the mechanism downstream of the prostaglandins remained unclear (Matsuoka et al., Proc. Nat. Acad. Sci. 100:4132-4137 (2003)). By using mice lacking each of the four prostaglandin receptors (EP₁-EP₄) and an EP₁-selective antagonist, Matsuoka et al. showed that both EP₁ and EP₃ are required for adrenocorticotropic hormone release in response to LPS. These and other findings indicated that EP₁- and EP₃-mediated neuronal pathways converge at corticotropin-releasing hormone-containing neurons in the paraventricular nucleus of the hypothalamus to induce HPA axis activation during disease.

Wild-type EP₁ receptor and EP₁ receptor variants can also be involved in kidney function. EP₁ receptor mRNA is expressed in the collecting duct of the kidney where its stimulation reduces NaCl and water absorption, promoting natriuresis and diuresis (Breyer and Breyer, Annu. Rev. Physiol. 63:579-605 (2001)). As disclosed herein, the EP₁ receptor variant EP₁ VAR-1 is also expressed in kidney (see FIG. 3).

Wild-type EP₁ receptor and EP₁ receptor variants can also be involved in colon carcinogenesis. For example, Mutoh et al. have shown that the EP₁ receptor, but not the EP₃ receptor, is involved in mouse colon carcinogenesis (Mutoh et al., Cancer Res. 62:28-32 (2002)). Wild-type EP₁ receptor and EP₁ receptor variants can be involved in these or other physiological processes.

The present invention relates to novel EP₁ receptor variants related to the wild-type EP₁ receptor. The EP₁ receptor has been cloned from several species including mouse (GenBank Accession No. NM_{—}013641.1), rat (GenBank Accession No. NM_{—}013100.1), and human (GenBank Accession No. NM_{—}000955.1).

Alternatively spliced isoforms of the EP₁ receptor have been identified in the rat (Okuda-Ashitaka et al., J. Biol. Chem. 271:31255-31261 (1996)). The deduced amino acid sequence of the first rat isoform to be cloned (rEP₁) extends for 88 amino acids beyond the splice site and includes TM6, TM7 and a carboxyl terminus consisting of 40 amino acids. The deduced amino acid sequence of the second rat EP₁ receptor isoform (rEP₁-v) continues for 49 amino acids beyond the splice site and completely lacks a cytoplasmic carboxyl terminus.

The invention provides novel EP₁ receptor variants including an alternatively spliced form of the wild-type human EP₁ receptor EP₁ VAR-1. In one embodiment, the invention provides an isolated polypeptide containing an amino acid sequence having at least 50% amino acid identity with SEQ ID NO: 8 and containing the amino acid sequence of SEQ ID NO: 4 or 6, or a conservative variant thereof. In further embodiments, the invention provides an isolated polypeptide containing an amino acid sequence having at least 80% or at least 90% amino acid identity with SEQ ID NO: 8 and containing the amino acid sequence of SEQ ID NO: 4 or 6, or a conservative variant thereof. In yet a further embodiment, the invention provides an isolated polypeptide containing the amino acid sequence of SEQ ID NO: 2, or a conservative variant thereof. In other embodiments, the invention provides an isolated polypeptide that contains or consists of the amino acid sequence of SEQ ID NO: 2.

The invention additionally provides a cell which includes an exogenously expressed polypeptide containing an amino acid sequence having at least 50% amino acid identity with SEQ ID NO: 8 and containing the amino acid sequence of SEQ ID NO: 4 or 6, or a conservative variant thereof; a cell which includes an exogenously expressed polypeptide containing the amino acid sequence of SEQ ID NO: 2, or a conservative variant thereof; or a cell which includes an exogenously expressed polypeptide containing or consisting of SEQ ID NO: 2.

The present invention also provides a method for identifying a compound that modulates an EP₁ receptor variant by contacting an isolated EP₁ receptor variant or an EP₁ receptor variant over-expressed in a genetically engineered cell with a compound and determining the level of an indicator which correlates with modulation of an EP₁ receptor variant, where an alteration in the level of the indicator as compared to a control level indicates that the compound is a compound that modulates the EP₁ receptor variant, and where the EP₁ receptor variant contains an amino acid sequence having at least 50% amino acid identity with SEQ ID NO: 8 and containing the amino acid sequence of SEQ ID NO: 4 or 6, or a conservative variant thereof. The alteration in the level of indicator can be, for example, an increase or decrease in the level of an indicator such as, without limitation, calcium. A method of the invention can be practiced with any of a variety of EP₁ receptor variants such as an isolated polypeptide containing an amino acid sequence having at least 80% or at least 90% amino acid identity with SEQ ID NO: 8 and containing the amino acid sequence of SEQ ID NO: 4 or 6, or a conservative variant thereof; or an isolated polypeptide containing the amino acid sequence of SEQ ID NO: 2, or a conservative variant thereof. A method of the invention also can be practiced using any of a variety of EP₁ receptor variants over-expressed in a genetically engineered cell. In one embodiment, the EP₁ receptor variant is exogenously over-expressed in the genetically engineered cell. A variety of compounds can be screened according to the methods of the invention including, but not limited to, polypeptides and small molecules.

The present invention further provides a method for identifying a compound that specifically binds to an EP₁ receptor variant by contacting an isolated EP₁ receptor variant or an EP₁ receptor variant over-expressed in a genetically engineered cell with a compound and determining specific binding of the compound to the EP₁ receptor variant, and where the EP₁ receptor variant contains an amino acid sequence having at least 50% amino acid identity with SEQ ID NO: 8 and containing the amino acid sequence of SEQ ID NO: 4 or 6, or a conservative variant thereof. In particular embodiments, a method of the invention is practiced using an isolated EP₁ receptor variant such as a polypeptide containing an amino acid sequence having at least 50% amino acid identity with SEQ ID NO: 8 and containing the amino acid sequence of SEQ ID NO: 4 or 6, or a conservative variant thereof; an isolated EP₁ receptor variant containing the amino acid sequence of SEQ ID NO: 2, or a conservative variant thereof; or an isolated EP₁ receptor variant containing or consisting of SEQ ID NO: 2. In another embodiment, a method of the invention is practiced using an EP₁ receptor variant over-expressed in a genetically engineered cell, for example, an EP₁ receptor variant exogenously over-expressed in a genetically engineered cell. In the methods of the invention, contacting can occur in vivo or in vitro, and the compounds to be screened can include, without limitation, polypeptides and small molecules.

The invention further provides a method for identifying a compound that differentially modulates an EP₁ receptor variant by a) contacting an isolated EP₁ receptor variant or an EP₁ receptor variant over-expressed in a genetically engineered cell with a compound; b) determining the level of an indicator which correlates with modulation of an EP₁ receptor variant; c) contacting a second receptor with the compound; d) determining the level of a corresponding indicator after contacting of the compound to the second receptor; and e) comparing the level of the indicator from step (b) with the level of the corresponding indicator from step (d), where a different level of the indicator from step (b) compared to the level of the corresponding indicator from step (d) indicates that the compound is a compound that differentially modulates the EP₁ receptor variant, and where the EP₁ receptor variant contains an amino acid sequence having at least 50% amino acid identity with SEQ ID NO: 8 and containing the amino acid sequence of SEQ ID NO: 4 or 6, or a conservative variant thereof. The second receptor can be, for example, a distinct EP₁ receptor variant or a wild-type EP₁ receptor from the same or a different species, or a functional fragment thereof. The level of the indicator from step (b) can be greater or less than the level of the indicator from step (d) and the indicator can be, for example, calcium. In particular embodiments, a method of the invention is practiced using an isolated EP₁ receptor variant such as a polypeptide containing an amino acid sequence having at least 50% amino acid identity with SEQ ID NO: 8 and containing the amino acid sequence of SEQ ID NO: 4 or 6, or a conservative variant thereof; an isolated EP₁ receptor variant containing the amino acid sequence of SEQ ID NO: 2, or a conservative variant thereof; or an isolated EP₁ receptor variant containing or consisting of SEQ ID NO: 2. In another embodiment, a method of the invention is practiced using an EP₁ receptor variant over-expressed in a genetically engineered cell, for example, an EP₁ receptor variant exogenously over-expressed in a genetically engineered cell. In the methods of the invention, the compounds to be screened can include, without limitation, polypeptides and small molecules.

The invention further provides a method for identifying a compound that differentially binds to an EP₁ receptor variant by a) contacting an isolated EP₁ receptor variant or an EP₁ receptor variant over-expressed in a genetically engineered cell with a compound; b) determining specific binding of the compound to the EP₁ receptor variant; c) contacting a second receptor with the compound; d) determining specific binding of the compound to the second receptor; and e) comparing the level of specific binding from step (b) with the level of specific binding from step (d), where a different level of specific binding from step (b) compared to the level of specific binding from step (d) indicates that the compound is a compound that differentially binds to the EP₁ receptor variant, and where the EP₁ receptor variant contains an amino acid sequence having at least 50% amino acid identity with SEQ ID NO: 8 and containing the amino acid sequence of SEQ ID NO: 4 or 6, or a conservative variant thereof. The second receptor can be, for example, a distinct EP₁ receptor variant or a wild-type EP₁ receptor from the same or a different species, or a functional fragment thereof. The different level of specific binding can be an increased or decreased level. In particular embodiments, a method of the invention is practiced using an isolated EP₁ receptor variant such as a polypeptide containing an amino acid sequence having at least 50% amino acid identity with SEQ ID NO: 8 and containing the amino acid sequence of SEQ ID NO: 4 or 6, or a conservative variant thereof; an isolated EP₁ receptor variant containing the amino acid sequence of SEQ ID NO: 2, or a conservative variant thereof; or an isolated EP₁ receptor variant containing or consisting of SEQ ID NO: 2. In another embodiment, a method of the invention is practiced using an EP₁ receptor variant over-expressed in a genetically engineered cell, for example, an EP₁ receptor variant exogenously over-expressed in a genetically engineered cell. In the methods of the invention, contacting can occur in vivo or in vitro, and the compounds to be screened can include, without limitation, polypeptides and small molecules.

The invention also provides an isolated nucleic acid molecule having a nucleotide sequence that encodes a polypeptide containing an amino acid sequence having at least 50% amino acid identity with SEQ ID NO: 8 and containing the amino acid sequence of SEQ ID NO: 4 or 6, or a conservative variant thereof. The invention also provides an isolated nucleic acid molecule having a nucleotide sequence that encodes a polypeptide containing an amino acid sequence having at least 80% or at least 90% amino acid identity with SEQ ID NO: 8 and containing the amino acid sequence of SEQ ID NO: 4 or 6, or a conservative variant thereof. The invention further provides an isolated nucleic acid molecule containing a nucleotide sequence that encodes the amino acid sequence of SEQ ID NO: 2, or a conservative variant thereof. The invention also provides an isolated nucleic acid molecule containing a nucleotide sequence that encodes SEQ ID NO: 2, such as the nucleotide sequence of SEQ ID NO: 1. The invention further provides a vector containing a nucleic acid molecule having a nucleotide sequence that encodes: a polypeptide containing an amino acid sequence having at least 50% amino acid identity with SEQ ID NO: 8 and containing the amino acid sequence of SEQ ID NO: 4 or 6, or a conservative variant thereof; a polypeptide containing the amino acid sequence of SEQ ID NO: 2, or a conservative variant thereof, or a polypeptide consisting of SEQ ID NO: 2. Host cells containing such vectors are further provided herein.

The invention relates, in part, to the identification of novel EP₁ receptor variants. As used herein, the term “EP₁ receptor variant” means a polypeptide containing an amino acid sequence that has at least 50% amino acid identity with the wild-type human EP₁ receptor SEQ ID NO: 8 and further containing the amino acid sequence of SEQ ID NO: 4 or 6, or a conservative variant of SEQ ID NO: 4 or 6. An EP₁ receptor variant can contain an amino acid sequence having, for example, at least 50% amino acid identity, at least 60% amino acid identity, at least 70% amino acid identity, at least 80% amino acid identity, at least 83% amino acid identity, at least 85% amino acid identity, at least 90% amino acid identity, at least 95% amino acid identity, or at least 98% amino acid identity with the wild-type human EP₁ receptor SEQ ID NO: 8. As a non-limiting example, an EP₁ receptor variant can contain an amino acid sequence having at least 83% amino acid identity with SEQ ID NO: 8 and containing the amino acid sequence of SEQ ID NO: 4 or 6 or a conservative variant of SEQ ID NO: 4 or 6.

Based on the above, it is understood that species homologs of EP₁ receptor variants that contain the amino acid sequence of SEQ ID NO: 4 or 6, or a conservative variant thereof are encompassed by the definition of EP₁ receptor variant as used herein. As non-limiting examples, an isolated polypeptide containing the amino acid sequence of SEQ ID NO: 2, or consisting of the amino acid sequence of SEQ ID NO: 2, is an EP₁ receptor variant of the invention.

An EP₁ receptor variant differs from the known wild-type human EP₁ receptor polypeptide by containing the amino acid sequence of SEQ ID NO: 4 or 6, or a conservative variant of such an amino acid sequence. As used herein in reference to a specified amino acid sequence such as SEQ ID NO: 4 or 6, a “conservative variant” is a sequence in which a first amino acid is replaced by another amino acid or amino acid analog having at least one biochemical property similar to that of the first amino acid; similar properties include, yet are not limited to, similar size, charge, hydrophobicity or hydrogen-bonding capacity.

As an example, a conservative variant can be a sequence in which a first uncharged polar amino acid is conservatively substituted with a second (non-identical) uncharged polar amino acid such as cysteine, serine, threonine, tyrosine, glycine, glutamine or asparagine or an analog thereof. A conservative variant also can be a sequence in which a first basic amino acid is conservatively substituted with a second basic amino acid such as arginine, lysine, histidine, 5-hydroxylysine, N-methyllysine or an analog thereof. Similarly, a conservative variant can be a sequence in which a first hydrophobic amino acid is conservatively substituted with a second hydrophobic amino acid such as alanine, valine, leucine, isoleucine, proline, methionine, phenylalanine or tryptophan or an analog thereof. In the same way, a conservative variant can be a sequence in which a first acidic amino acid is conservatively substituted with a second acidic amino acid such as aspartic acid or glutamic acid or an analog thereof; a sequence in which an aromatic amino acid such as phenylalanine is conservatively substituted with a second aromatic amino acid or amino acid analog, for example, tyrosine; or a sequence in which a first relatively small amino acid such as alanine is substituted with a second relatively small amino acid or amino acid analog such as glycine or valine or an analog thereof. It is understood that a conservative variant of SEQ ID NO: 2, 4 or 6 can have one, two, three, four, five, ten, or more amino acid substitutions relative to the specified sequence and that such a conservative variant can include naturally and non-naturally occurring amino acid analogs.

It is understood that a fragment of an EP₁ receptor variant containing the amino acid sequence of SEQ ID NO: 6 can be useful in a method of the invention. As non-limiting examples, a functional fragment of an EP₁ receptor variant such as a ligand-binding fragment or a fragment of an EP₁ receptor variant that is involved in signal transduction can be useful in a method of the invention in place of the full-length EP₁ receptor variant. As further understood by one skilled in the art, an EP₁ receptor variant can optionally include non-homologous amino acid sequence. As non-limiting examples, an EP₁ receptor variant can contain an epitope tag or can be fused to a non-homologous polypeptide such as gluthionine S-transferase.

As discussed above, the EP₁ receptor variant EP₁ VAR-1 contains an amino acid sequence that is not present in the wild-type EP₁ receptor SEQ ID NO: 8 (see FIG. 2). The alternatively spliced EP₁ receptor variant EP₁ VAR-1 contains unique carboxy terminal amino acid sequence disclosed herein as SEQ ID NO: 4. Furthermore, a ten amino acid sequence spanning the junction between conserved exon 2 and the retained intron A present in EP₁ receptor variant EP₁ VAR-1 is disclosed herein as SEQ ID NO: 6. The ten amino acid sequence begins with four amino acid residues that correspond to amino acid sequence present in conserved exon 2 and further includes six amino acid residues derived from retained intron A present in EP₁ receptor variant EP₁ VAR-1. Thus, the invention provides an isolated polypeptide containing the amino acid sequence of SEQ ID NO: 4 or 6. The invention further provides an isolated polypeptide containing an amino acid sequence having at least 50% amino acid identity with SEQ ID NO: 8 and containing the amino acid sequence of SEQ ID NO: 4 or 6, or a conservative variant thereof. As non-limiting examples, the invention provides an isolated polypeptide containing the amino acid sequence of SEQ ID NO: 2 or a conservative variant thereof, such as an isolated polypeptide containing or consisting of the amino acid sequence of SEQ ID NO: 2.

Further provided herein is an isolated polypeptide containing or consisting of substantially the same amino acid sequence as SEQ ID NO: 2. The term “substantially the same,” when used herein in reference to an amino acid sequence, means a polypeptide having a similar, non-identical sequence that is considered by those skilled in the art to be a functionally equivalent amino acid sequence. An amino acid sequence that is substantially the same as a reference amino acid sequence can have at least 70%, at least 80%, at least 90%, or at least 95% or more identity to the reference sequence. The term substantially the same amino acid sequence also includes sequences encompassing, for example, modified forms of naturally occurring amino acids such as D-stereoisomers, non-naturally occurring amino acids, amino acid analogs and mimetics, so long as the polypeptide containing such a sequence retains a functional activity of the reference EP₁ receptor variant. A functional activity of an EP₁ receptor variant of the invention can be, for example, the ability to bind a compound such as, but not limited to, PGE₂, sulprostone, and iloprost, or the ability to initiate a particular intracellular signal transduction pathway.

It is understood that minor modifications in primary amino acid sequence can result in a polypeptide that has a substantially equivalent function as compared to a polypeptide of the invention. These modifications can be deliberate, as through site-directed mutagenesis, or may be accidental such as through spontaneous mutation. For example, it is understood that only a portion of the entire primary structure of an EP₁ receptor variant can be required in order to bind to compound such as PGE₂. Moreover, fragments of an EP₁ receptor variant of the invention containing the amino acid sequence of SEQ ID NO: 6 similarly are included within the definition of substantially the same amino acid sequence as long as at least one biological function of the EP₁ receptor variant is retained. It is understood that various molecules can be attached to an EP₁ receptor variant or other polypeptide of the invention. These molecules include, without limitation, heterologous polypeptides, carbohydrates, lipids, or chemical moieties such as radioactive or fluorescent label moieties.

The invention further provides an EP₁ receptor variant binding agent which binds the amino acid sequence of SEQ ID NO: 4, or an epitope thereof. As discussed above, SEQ ID NO: 4 represents the unique carboxy terminal amino acid sequence of alternatively spliced EP₁ receptor variant EP₁ VAR-1. An EP₁ receptor variant binding agent of the invention can be, without limitation, an antibody or antigen binding fragment thereof which binds the amino acid sequence of SEQ ID NO: 4, or an epitope thereof.

As used herein, the term “EP₁ receptor variant binding agent” means a molecule, such as a simple or complex organic molecule, carbohydrate, peptide, peptidomimetic, protein, glycoprotein, lipoprotein, lipid, nucleic acid molecule, antibody, aptamer or the like that specifically binds the unique EP₁ receptor variant carboxy-terminal amino acid sequence disclosed herein as SEQ ID NO: 4, or an epitope thereof. It is understood that such a binding agent does not specifically bind to a wild-type EP₁ receptor such as SEQ ID NO: 8 since a wild-type EP₁ receptor does not contain the unique carboxy terminal amino acid sequence disclosed herein as SEQ ID NO: 4.

An EP₁ receptor variant binding agent of the invention can be a polypeptide that specifically binds with high affinity or avidity to SEQ ID NO: 4, without substantial cross-reactivity to other unrelated sequences. The affinity of an EP₁ receptor variant binding agent of the invention generally is greater than about 10⁻⁴ M and can be greater than about 10⁻⁶ M, typically being in the range of 10⁻⁴ M to 10⁻¹⁰ M. An EP₁ receptor variant binding agent of the invention can bind, for example, with high affinity such as an affinity of 10⁻⁷ M to 10⁻¹⁰ M. Specific examples of binding agents of the invention include, but are not limited to, polyclonal and monoclonal antibodies that specifically bind an epitope within SEQ ID NO: 4; and nucleic acid molecules, nucleic acid analogs, and small organic molecules, identified, for example, by affinity screening of a nucleic acid or small molecule library against SEQ ID NO: 4. For certain applications, an EP₁ receptor variant binding agent can be utilized that preferentially recognizes a particular conformational or post-translationally modified state of SEQ ID NO: 4. It is understood that an EP₁ receptor variant binding agent of the invention can be labeled with a detectable moiety, if desired, or rendered detectable by specific binding to a detectable secondary agent.

In one embodiment, an EP₁ receptor variant binding agent of the invention is an antibody or antigen-binding fragment thereof. As used herein, the term “antibody” is used in its broadest sense to mean a polyclonal or monoclonal antibody or an antigen binding fragment of such an antibody. Such an antibody of the invention is characterized by having specific binding activity for SEQ ID NO: 4, or an epitope thereof, of at least about 1×10⁵ M⁻¹. Thus, Fab, F(ab′)₂, Fd and Fv fragments of an antibody, which retain specific binding activity for SEQ ID NO: 4, or an epitope thereof, are included within the definition of antibody as used herein. Specific binding activity can be readily determined by one skilled in the art, for example, by comparing the binding activity of the antibody to SEQ ID NO: 4, versus a control sequence. Methods of preparing polyclonal or monoclonal antibodies are well known to those skilled in the art. See, for example, Harlow and Lane, Antibodies: A Laboratory Manual, Cold Spring Harbor Laboratory Press (1988).

It is understood that the term antibody includes naturally occurring antibodies as well as non-naturally occurring antibodies such as, without limitation, single chain antibodies, chimeric, bi-functional and humanized antibodies, and antigen-binding fragments thereof. Such non-naturally occurring antibodies can be constructed using solid phase peptide synthesis, produced recombinantly or obtained, for example, by screening combinatorial libraries consisting of variable heavy chains and variable light chains as described in Huse et al., Science 246:1275-1281 (1989). These and other methods of making, for example, chimeric, humanized, CDR-grafted, single chain, and bi-functional antibodies are well known to those skilled in the art (Winter and Harris, Immunol. Today 14:243-246 (1993); Ward et al., Nature 341:544-546 (1989); Harlow and Lane, supra, 1988; Hilyard et al., Protein Enqineerinq: A Practical approach (IRL Press 1992); and Borrabeck, Antibody Engineering, 2d ed. (Oxford University Press 1995)).

An antibody of the invention can be prepared using as an antigen a polypeptide or peptide containing SEQ ID NO: 4, or an epitope thereof, which can be prepared, for example, from natural sources, produced recombinantly, or chemically synthesized. Such a polypeptide or peptide is a functional antigen if the polypeptide or peptide can be used to generate an antibody that specifically binds SEQ ID NO: 4, or an epitope thereof. As is well known in the art, a non-antigehic or weakly antigenic polypeptide or peptide can be made antigenic by coupling the polypeptide or peptide to a carrier molecule such as bovine serum albumin (BSA) or keyhole limpet hemocyanin (KLH). Various other carrier molecules and methods for coupling a polypeptide or peptide to a carrier molecule are well known in the art (see, for example, Harlow and Lane, supra, 1988). An antigenic polypeptide or peptide can also be generated by expressing the polypeptide or peptide as a fusion protein, for example, fused to glutathione S transferase, polyHis or the like. Methods for expressing polypeptide fusions are well known to those skilled in the art as described, for example, in Ausubel et al., Current Protocols in Molecular Biology (Supplement 47), John Wiley & Sons, New York (1999).

The present invention also provides a cell that includes an exogenously expressed polypeptide containing the amino acid sequence of SEQ ID NO: 4 or 6. Further provided herein is a cell that includes an exogenously expressed polypeptide containing an amino acid sequence having at least 50% amino acid identity with SEQ ID NO: 8, and containing the amino acid sequence of SEQ ID NO: 4 or 6, or a conservative variant thereof. The invention provides, for example, a cell that includes an exogenously expressed polypeptide containing the amino acid sequence of SEQ ID NO: 2, or a conservative variant thereof.

Such a cell containing an exogenously expressed polypeptide of the invention can be generated by expressing a nucleic acid molecule encoding the polypeptide in a suitable host cell, such as a bacterial cell, yeast cell, insect cell, oocyte or other amphibian cell, or mammalian cell, using methods well known in the art. Suitable expression vectors are well known in the art and include vectors in which a nucleic acid molecule is operatively linked to a regulatory element such as a promoter or enhancer region that is capable of regulating expression of a linked nucleic acid molecule. Appropriate expression vectors include, without limitation, those that can be replicated in eukaryotic or prokaryotic cells, those that remain episomal as well as those which integrate into the host cell genome, and those including constitutive, inducible or regulated promoters, enhancers or other regulatory elements.

Suitable expression vectors for prokaryotic or eukaryotic cells are well known to those skilled in the art (see, for example, Ausubel et al., supra, 1999). Eukaryotic expression vectors can contain, for example, a regulatory element such as, but not limited to, the SV40 early promoter, the cytomegalovirus (CMV) promoter, the mouse mammary tumor virus (MMTV) steroid-inducible promoter, the Moloney murine leukemia virus (MMLV) promoter, and the like. One skilled in the art will know or can readily determine an appropriate expression vector for a particular host cell.

Useful expression vectors optionally contain a regulatory element that provides cell or tissue specific expression or inducible expression of the operatively linked nucleic acid molecule. One skilled in the art can readily determine an appropriate tissue-specific promoter or enhancer that allows expression of a polypeptide of the invention in a desired tissue. Furthermore, any of a variety of inducible promoters or enhancers can also be included in an expression vector for regulated expression of a polypeptide of the invention. Such inducible systems include, yet are not limited to, a tetracycline inducible gene regulatory region (Gossen & Bijard, Proc. Natl. Acad. Sci. USA, 89:5547-5551 (1992); Gossen et al., Science, 268:1766-1769 (1995); Clontech, Palo Alto, Calif.); a metallothionein promoter inducible by heavy metals; an insect steroid hormone responsive gene regulatory region responsive to ecdysone or related steroids such as muristerone (No et al., Proc. Natl. Acad. Sci. USA, 93:3346-3351 (1996); Yao et al., Nature, 366:476-479 (1993); Invitrogen, Carlsbad, Calif.); a mouse mammary tumor virus (MMTV) gene regulatory region induced by steroids such as glucocortocoid and estrogen (Lee et al., Nature, 294:228-232 (1981); and a heat shock promoter.

An expression vector useful in the invention can be a viral vector such as, without limitation, a retrovirus, adenovirus, adeno-associated virus, lentivirus, or herpesvirus vector. Viral based systems provide the advantage of being able to introduce relatively high levels of a heterologous nucleic acid molecule into a variety of cells. Additionally, certain viral vectors can introduce heterologous DNA into non-dividing cells. A variety of suitable viral expression vectors are well known in the art and include, without limitation, herpes simplex virus vectors (U.S. Pat. No. 5,501,979), vaccinia virus vectors (U.S. Pat. No. 5,506,138), cytomegalovirus vectors (U.S. Pat. No. 5,561,063), modified Moloney murine leukemia virus vectors (U.S. Pat. No. 5,693,508), adenovirus vectors (U.S. Pat. Nos. 5,700,470 and 5,731,172), adeno-associated virus vectors (U.S. Pat. No. 5,604,090), constitutive and regulatable retrovirus vectors (U.S. Pat. Nos. 4,405,712; 4,650,764 and 5,739,018, respectively), papilloma virus vectors (U.S. Pat. Nos. 5,674,703 and 5,719,054), and the like.

A cell can be generated that transiently or stably expresses an exogenously expressed polypeptide of the invention. Expression vectors for transient or stable expression of a polypeptide of the invention can be introduced into cells using transfection methods well known to one skilled in the art. Such methods include, without limitation, infection using viral vectors, lipofection, electroporation, particle bombardment and transfection such as calcium-phosphate mediated transfection. Detailed procedures for these methods can be found in Sambrook et al., Molecular Cloning: A Laboratory Manual Cold Spring Harbor Laboratory Press (1989), and the references cited therein. Useful mammalian expression vectors and methods of introducing such vectors into mammalian cells either ex vivo or in vivo are well known in the art. As non-limiting examples, a plasmid expression vector can be introduced into a cell by calcium-phosphate mediated transfection, DEAE dextran-mediated transfection, lipofection, polybrene- or polylysine-mediated transfection, electroporation, or by conjugation to an antibody, gramacidin S, artificial viral envelope or other intracellular carrier. A viral expression vector can be introduced into a cell by infection or transduction, for example, or by encapsulation in a liposome. It further is understood that polypeptides can be delivered directly into cells using a lipid-mediated delivery system (Zelphati et al., J. Biol. Chem. 276:35103-35110 (2001)) to produce a cell that contains an exogenously expressed polypeptide of the invention.

Exemplary host cells that can be used to exogenously express a polypeptide of the invention include, yet are not limited to, mammalian primary cells; established mammalian cell lines such as COS, CHO, HeLa, NIH3T3, HEK 293, and HEK 293/EBNA cells; amphibian cells such as Xenopus embryos and oocytes; and other vertebrate cells. Exemplary host cells further include, without limitation, insect cells such as Drosophila, Spodoptera frugiperda and other cells compatible with baculovirus expression systems (Murakimi et al., Cytokine, 13:18-24 (2001)); yeast cells such as Saccharomyces cerevisiae, Saccharomyces pombe, or Pichia pastoris; and prokaryotic cells such as Escherichia coli. Following transfection, cells exogenously expressing a polypeptide of the invention can be selected, for example, using drug resistance. A quantitative assay such as, for example, immunoblot analysis, immunoprecipitation or ELISA can determine the amount of a polypeptide of the invention expressed in a transfected cell. Such methods are known to one skilled in the art and can be found, for example, in Ausubel et al., supra, 1989, or in Harlow et al., supra, 1988.

Further provided herein are methods for identifying a compound that modulates an EP₁ receptor, identifying a compound that differentially modulates an EP₁ receptor, identifying a compound that specifically binds an EP₁ receptor, and identifying a compound that differentially binds to an EP₁ receptor. In particular, the invention provides a method for identifying a compound that modulates an EP₁ receptor variant by contacting an EP₁ receptor variant with a compound and determining the level of an indicator which correlates with modulation of an EP₁ receptor variant, where an alteration in the level of the indicator as compared to a control level indicates that the compound is a compound that modulates the EP₁ receptor variant. Further provided herein are methods for identifying a compound that modulates an EP₁ receptor variant by contacting an isolated EP₁ receptor variant or an EP₁ receptor variant over-expressed in a genetically engineered cell with a compound and determining the level of an indicator which correlates with modulation of an EP₁ receptor variant, where an alteration in the level of the indicator as compared to a control level indicates that the compound is a compound that modulates the EP₁ receptor variant.

As used herein in reference to an EP₁ receptor variant, the term “modulates” means the ability to alter a characteristic of an EP₁ receptor variant. A characteristic of an EP₁ receptor variant that can be altered can include, without limitation, an amount, activity, or physical conformation of an EP₁ receptor variant. As a non-limiting example, a compound that modulates an EP₁ receptor variant can increase or decrease the binding of an EP₁ receptor variant to a ligand such as, without limitation, PGE₂; sulprostone; iloprost; 16, 16-dimethyl PGE₂; 17-phenyl PGE₂, for example, 17-phenyl-ω-trinor PGE₂; carbacyclin; ICI 80205 (Lawrence et al., Br. J. Pharmacol. 105:271-278 (1992)); 17-phenyl-PGF_{2α}; ZK110841; or Enprostil, and above (Ungrin et al., Mol. Pharmacology 59:1446-1456 (2001)). Also, for example, a compound can increase or decrease the binding of an EP₁ receptor variant to an intracellular signaling molecule that initiates a signal transduction pathway within a cell. It is understood that compounds that modulate an EP₁ receptor variant include compounds that specifically bind to an EP₁ receptor variant as well as compounds that do not specifically bind to an EP₁ receptor variant.

A method of the invention for identifying a compound that modulates an EP₁ receptor variant involves determining the level of an indicator which correlates with modulation of an EP₁ receptor variant, where an alteration in the level of the indicator as compared to a control level indicates that the compound modulates the EP₁ receptor variant. As used herein, the term “indicator” means a detectable substance which is altered qualitatively or quantitatively in response to modulation of an EP₁ receptor variant. An indicator can be a substance that is normally present in a cell such as a signal transduction molecule, or a substance that is exogenously expressed or otherwise added to a cell, the level of which correlates with modulation of an EP₁ receptor variant. One example of an indicator is luciferase. Signal transduction molecules are intracellular substances such as, without limitation, cyclic AMP, inositol phosphates and calcium, the level of which can be altered in response to modulation of an EP₁ receptor variant.

As understood by those of skill in the art, assay methods for identifying compounds that modulate an EP₁ receptor variant generally require comparison to a control. For example, in a method of the invention an alteration in the level of an indicator which correlates with modulation of an EP₁ receptor variant is compared to a control level of the indicator. One type of a control is a sample that is treated substantially the same as the EP₁ receptor variant which is contacted with a compound, with the distinction that the control sample is not exposed to the compound. Controls include, but are not limited to, historical reference values, and samples that are assayed simultaneously or sequentially in comparison to the EP₁ receptor variant which is contacted with the compound.

In one embodiment, a method of the invention is practiced using calcium as the indicator. For example, as disclosed herein in Example III, a FLIPR assay can be used to identify compounds that modulate an EP₁ receptor variant by determining the level of calcium that results after contacting a receptor with a compound. Exogenously expressed substances such as, for example, luciferase, β-galactosidase and green fluorescent protein (GFP) also can be indicators useful in a method of the invention (see Example III).

Further provided herein are methods for identifying a compound that specifically binds to an EP₁ receptor variant by contacting an EP₁ receptor variant with a compound and determining specific binding of the compound to the EP₁ receptor variant. Additionally provided herein are methods for identifying a compound that specifically binds to an EP₁ receptor variant by contacting an isolated EP₁ receptor variant or an EP₁ receptor variant over-expressed in a genetically engineered cell with a compound and determining specific binding of the compound to the EP₁ receptor variant.

As used herein in reference to a compound and an EP₁ receptor variant, the term “specific binding” means binding with an affinity for the target EP₁ receptor variant that is measurably higher than the affinity for an unrelated polypeptide such as an unrelated G protein coupled receptor such as a rhodopsin receptor. For example, a polypeptide or small molecule compound that specifically binds an EP₁ receptor variant has an affinity for the EP₁ receptor variant that is measurably higher than its affinity for an unrelated polypeptide. Binding affinity can be low or high so long as the binding is sufficient to be detectable. For example, a compound can specifically bind an EP₁ receptor variant with a binding affinity (Kd) of about 10⁻⁴ M or less, 10⁻⁵ M or less, 10⁻⁶ M or less, 10⁻⁷ M or less, 10⁻⁸ M or less, or 10⁻⁹ M or less. Several methods for detecting or measuring specific binding are well known in the art and discussed further below.

The screening methods of the invention can be practiced, for example, using an EP₁ receptor variant over-expressed in a genetically engineered cell. As used herein, the term “genetically engineered cell” means a cell having genetic material which is altered by the hand of man. Such a cell can contain a transient or permanent alteration of its genetic material including, for example, alteration in genomic or episomal genetic material. The genetic material in a genetically engineered cell can be altered using, without limitation, an exogenously expressed nucleic acid molecule, chemical mutagen or transposable element. It is understood that a genetically engineered cell can contain one or more man-made alterations, for example, a cell can be co-transfected with more than one expression vector. As used herein in relation to an EP₁ receptor variant in a genetically engineered cell, the term “over-expressed” means having a protein level of an EP₁ receptor variant greater than the level seen in a corresponding non-genetically engineered cell.

As understood by one skilled in the art, an EP₁ receptor variant can be over-expressed in a genetically engineered cell, for example, by exogenously expressing a nucleic acid molecule encoding the EP₁ receptor variant in a cell as described herein above. It is further understood that an EP₁ receptor variant can be over-expressed in a cell that does not normally express the EP₁ receptor variant, or in a cell that naturally expresses the endogenous EP₁ receptor variant. As a non-limiting example, an EP₁ receptor variant can be over-expressed in a cell that expresses endogenous EP₁ receptor variant at a low level. In addition, an EP₁ receptor variant can be over-expressed in a genetically engineered cell, for example, by expressing a regulatory molecule in the cell to increase expression of the endogenous EP₁ receptor variant. Another example of a method whereby an EP₁ receptor variant can be over-expressed in a genetically engineered cell is recombination of a heterologous regulatory region such as, without limitation, a promoter, enhancer or 3′ regulator, in the cell such that the heterologous regulatory region results in over-expression of endogenous EP₁ receptor variant. As understood by one skilled in the art, over-expression of an EP₁ receptor variant in a genetically engineered cell includes, without limitation, over-expression of the variant on the surface of the cell, within a cell membrane or in the cytosolic portion of the cell.

An EP₁ receptor variant also can be over-expressed in a cell using a chemical agent. Thus, the invention provides a method for identifying a compound that modulates an EP₁ receptor variant by contacting the EP₁ receptor variant with a compound, where the EP₁ receptor variant is over-expressed in a cell using a chemical agent, and determining the level of an indicator which correlates with modulation of an EP₁ receptor variant, where an alteration in the level of the indicator as compared to a control level indicates that the compound is a compound that modulates the EP₁ receptor variant. The invention also provides a method for identifying a compound-that specifically binds to an EP₁ receptor variant by contacting the EP₁ receptor variant with a compound, where the EP₁ receptor variant is over-expressed in a cell using a chemical agent, and determining specific binding of the compound to the EP₁ receptor variant. Chemical agents that can result in over-expression of an EP₁ receptor variant can include, without limitation, chemicals that induce the level or activity of regulatory factor, such as a transcription factor, that is involved in EP₁ receptor variant expression.

As described above, the methods of the invention can be practiced with a cell that over-exptesses an EP₁ receptor variant. In addition, it is understood that an extract of a cell that over-expresses an EP₁ receptor variant such as a genetically engineered cell that over-expresses an EP₁ receptor variant can be useful in the methods of the invention. Methods for generating different types of cellular extracts including, without limitation, whole cell extracts, membrane extracts, cytosolic extracts and nuclear extracts are well known in the art. As a non-limiting example, receptor enriched plasma membrane fractions can be obtained by continuous or discontinuous gradients of, for example, sucrose as described in Woodward and Lawrence, Biochemical Pharmacology 47:1567-1674 (1994).

Isolated EP₁ receptor variants also can be useful in the screening methods of the invention. As used herein in reference to an EP₁ receptor variant, the term “isolated” means the EP₁ receptor variant is substantially separated from other polypeptides. For example, an isolated EP₁ receptor variant derived from a cell can be substantially purified away from other polypeptides in the cell. An isolated EP₁ receptor variant can contain non-polypeptide components, for example, an isolated EP₁ receptor variant can be associated with a natural or artificial lipid containing membrane. In one embodiment, a method of the invention is practiced with an isolated EP₁ receptor variant that contains an amino acid sequence having at least 50% amino acid identity with SEQ ID NO: 8 and containing the amino acid sequence of SEQ ID NO: 4 or 6, or a conservative variant thereof. In another embodiment, a method of the invention is practiced with an isolated EP₁ receptor variant that contains an amino acid sequence having at least 80% or at least 90% amino acid identity with SEQ ID NO: 8 and containing the amino acid sequence of SEQ ID NO: 4 or 6, or a conservative variant thereof. In a further embodiment, a method of the invention is practiced with an EP₁ receptor variant that contains the amino acid sequence of SEQ ID NO: 2, or a conservative variant thereof. In a yet further embodiment, a method of the invention is practiced with an isolated EP₁ receptor variant that contains or consists of SEQ ID NO: 2.

An EP₁ receptor variant of the invention can be prepared in isolated form using conventional biochemical purification methods, starting either from tissues containing the desired EP₁ receptor variant or from recombinant sources. An EP₁ receptor variant can be isolated by any of a variety of methods well-known in the art, including, but not limited to, precipitation, gel filtration, ion-exchange, reverse-phase and affinity chromatography, and combinations thereof. Other well-known methods for protein isolation are described in Deutscher et al., Guide to Protein Purification: Methods in Enzymology Vol. 182, (Academic Press, (1990)). Methods suitable for isolating an EP₁ receptor variant of the invention using biochemical purification are known in the art as described for example, in Venter and Harrison, (eds), Receptor Purification Procedures (Liss, (1984)); Litwack, Receptor Purification: Receptors for CNS Agents, Growth Factors, Hormones, & Related Substances, (Humana Press, (1990)); or Litwack, Receptor Purification: Receptors for Steroid Hormones, Thyroid Hormones, Water Balancing Hormone, & Others, (Humana Press, (1990)). Purification of the receptor variant can be routinely monitored, for example, by an immunological assay or functional assay such as a ligand binding assay.

An isolated EP₁ receptor variant of the invention also can be produced by chemical synthesis. As a non-limiting example, synthetic isolated EP₁ receptor variants, including fragments thereof, can be produced using an Applied Biosystems, Inc. Model 430A or 431A automatic peptide synthesizer (Foster City, Calif.) employing the chemistry provided by the manufacturer. Methods for synthesizing isolated polypeptides are well known in the art (see, for example, Bodanzsky, Principles of Peptide Synthesis (1st ed. & 2d rev. ed.), Springer-Verlag, New York, N.Y. (1984 & 1993), see Chapter 7; Stewart and Young, Solid Phase Peptide Synthesis, (2d ed.), Pierce Chemical Co., Rockford, Ill. (1984)).

In the methods of the invention for identifying a compound that modulates, or specifically binds to, an EP₁ receptor variant, an isolated EP₁ receptor variant or EP₁ receptor variant over-expressed in a genetically engineered cell can be contacted with a compound in a solution under conditions suitable for interaction between the EP₁ receptor variant and compound. Such contact can occur in vitro, such as in an isolated cell in cell culture, in a whole or partially purified cell extract, or with an isolated polypeptide. As used herein, the term “in vitro” means in an artificial environment outside of a living organism or cell. Assays performed in a test tube, microcentrifuge tube, 96 well plate, 384 well plate, 1536 well plate or other assay format outside of an organism or living cell are in vitro assays. Experiments performed in cells or tissues that have been fixed and are therefore dead (sometimes referred to as in situ experiments) or using cell-free extracts from cells are in vitro. Contact can also occur in vivo using, for example, whole animals.

Conditions suitable for contacting an isolated EP₁ receptor variant or EP₁ receptor variant over-expressed in a genetically engineered cell with a compound are dependent on the characteristics of the EP₁ receptor variant and the compound. For example, the overall charge of the EP₁ receptor variant and the compound can be considered when adjusting the salt concentration or pH of a buffering solution to optimize the specific binding or modulation of the EP₁ receptor variant by the compound. Usually a salt concentration and pH in the physiological range, for example, about 100 mM KCl and pH 7.0 are reasonable starting points. In addition, other components such as glycerol or protease inhibitors can be added to the solution, for example, to inhibit polypeptide degradation. It is understood that the stability of the contact between the EP₁ receptor variant and the compound can be effected by the temperature at which such contact occurs and that the optimal temperature for contact can be routinely determined by those skilled in the art. For example, reactions can be performed on ice (4° C.), at room temperature (about 25° C.) or at body temperature (37° C.) Suitable conditions can be similar or identical to conditions used for binding of a compound to the wild-type human EP₁ receptor. Such conditions are known in the art and include, for example, contact in a binding buffer containing 10 mM potassium phosphate (pH 6.0), 1 mM EDTA, and 10 mM MgCl₂, and incubation at room temperature for one hour, as described in Funk et al., supra, 1993.

The screening methods of the invention are useful for identifying compounds that modulate or differentially modulate, or that specifically or differentially bind an EP₁ receptor variant. As used herein, the term “compound” means a molecule of natural or synthetic origin. A compound can be, without limitation, a small organic or inorganic molecule, polypeptide, peptide, peptidomimetic, non-peptidyl compound, carbohydrate, lipid, antibody or antibody fragment, aptamer, or nucleic acid molecule. In one embodiment, the compound is a small organic molecule. It is understood that a compound can have a known or unknown structure, and can be assayed as an isolated molecule or as part of a population of compounds such as a library.

As understood by one skilled in the art, a compound can specifically bind to an EP₁ receptor variant without modulating the EP₁ receptor variant; specifically bind to an EP₁ receptor variant, thereby modulating the EP₁ receptor variant; or modulate an EP₁ receptor variant without specifically binding the EP₁ receptor variant. Compounds that specifically bind to an EP₁ receptor variant can include, without limitation, PGE₂; prostanoid-like compounds; and non-prostanoid-like structures identified as EP₁ receptor ligands, for example, by screening of chemical libraries. A compound that modulates an EP₁ receptor variant but does not directly bind to the EP₁ receptor variant can be, for example, a compound that binds to or effects the activity of a polypeptide in a cell, where that polypeptide increases or decreases the level of an EP₁ receptor variant. Such polypeptides include, without limitation, transcription or translation regulatory factors, signal transduction polypeptides; kinases and phosphatases; polypeptides that bind to an EP₁ receptor variant; and anti-sense oligonucleotides, inhibitor RNA molecules and ribozymes, which act on the nucleic acid that encodes the EP₁ receptor variant.

Compounds that modulate or specifically bind to an EP₁ receptor variant further include, but are not limited to, agonists and antagonists. An agonist can be a compound that binds to a receptor and activates it, producing a pharmacological response such as contraction, relaxation, secretion, or enzyme activation. An antagonist is a compound which can attenuate the effect of an agonist. An antagonist can be competitive, meaning it binds reversibly to a region of the receptor in common with an agonist, but occupies the site without activating the effector mechanism. The effects of a competitive antagonist can be overcome by increasing the concentration of agonist, thereby shifting the equilibrium and increasing the proportion of receptors occupied by agonist. Alternatively, antagonists can be non-competitive, where no amount of agonist can completely overcome the inhibition once it has been established. Non-competitive antagonists can bind covalently to the agonist binding site (called competitive irreversible antagonists), in which case there is a period before the covalent bond forms during which competing ligands can prevent the inhibition. Other types of non-competitive antagonists act allosterically at a different site on the receptor.

Other classes of compounds that can modulate or specifically bind to an EP₁ receptor variant include inverse agonists, which are compounds which produce an opposite physiological effect to that of an agonist, yet act at the same receptor. Such compounds have also been described as negative antagonists, or as having negative efficacy. Another class of compounds that can modulate or specifically bind to an EP₁ receptor variant is partial agonists, which are agonists that are unable to produce maximal activation of the receptor.

A library of compounds can be useful in the screening methods of the invention. Such a library can be a random collection of compounds or a focused collection of compounds, for example, compounds that are rationally designed or pre-selected based on physical or functional characteristics. For example, a library of prostanoids or prostanoid-related compounds can be useful in the screening methods of the invention. Libraries useful in the methods of the invention include, yet are not limited to, natural product libraries derived from, without limitation, microorganisms, animals, plants, and marine organisms; combinatorial chemical or other chemical libraries such as those containing randomly synthesized compounds; combinatorial libraries containing structural analogs of prostanoids or other known compounds, or random or biased assortments of, for example, small organic molecules, polypeptides, oligonucleotides, and combinations thereof. Still other libraries of interest include peptidomimetic, multiparallel synthetic collection, and recombinatorial libraries. Combinatorial and other chemical libraries are known in the art, as described, for example, in Myers, Curr. Opin. Biotechnol. 8:701-707 (1997). Appropriate libraries can be assembled from catalog sources such as Cayman Chemical Co. (Ann Arbor, Mich.), BIOMOL Research Laboratories, Inc. (Plymouth Meeting, Pa.), Tocris Cooksoon Inc. (Ellisville, Mo.), and others. These libraries can include, without limitation, fatty acids, fatty acid amides and esters, and eicosanoids.

In a screening method of the invention, the members of a library of compounds can be assayed for activity individually, in pools, or en masse. An example of en masse screening to identify a compound that modulates or specifically binds to an EP₁ receptor variant is as follows: a library of compounds is assayed in pools for the ability to modulate or specifically bind an EP₁ receptor variant; the sub-population which modulates or specifically binds the EP₁ receptor variant is subdivided; and the assay is repeated as needed in order to isolate an individual compound or compounds from the library that modulate or specifically bind the EP₁ receptor variant.

The methods of the invention can utilize high throughput screening (HTS) techniques to identify compounds that modulate or specifically bind to an EP₁ receptor variant. HTS assays permit screening of large numbers of compounds in an efficient manner. Cell-based high throughput screening systems include, but are not limited to, melanophore assays, yeast-based assay systems, and mammalian cell expression systems (Jayawickreme and Kost, Curr. Opin. Biotechnol. 8:629-634 (1997)). Automated and miniaturized high throughput screening assays are also useful in the methods of the invention (Houston and Banks, Curr. Opin. Biotechnol. 8:734-740 (1997)). High throughput screening assays are designed to identify “hits” or “lead compounds” having the desired modulating or specific binding activity, from which modified compounds can be prepared to improve a property of the initial lead compound. Chemical modification of the “hit” or “lead compound” can be based on an identifiable structure/activity relationship (SAR) between the “hit” and an EP₁ receptor variant of the invention. It is understood that assays such as the melanophore and radioligand binding assays disclosed below, and the FLIPR and luciferase assays disclosed in Example III, can be performed as conventional or high through-put screening assays to identify a compound that modulates or specifically binds to an EP₁ receptor variant, according to a method of the invention.

Various types of assays can be useful for identifying a compound that modulates or specifically binds to an EP₁ receptor variant in a method of the invention. For example, several assays can be used to measure specific binding of a compound to an EP₁ receptor variant in a method of the invention. A classic assay used for measuring specific binding of a compound to a receptor is a radioligand binding assay. Radioligand binding assays can be performed on cells or in solution, for example, using isolated cell membranes. As a non-limiting example, cells or cell membranes that transiently or stably over-express an EP, receptor variant can be incubated with a ligand including a novel or known ligand such as radioactively labeled PGE₂. After washing away any unbound radioactively labeled PGE₂, compounds of interest can be incubated with the cells. After incubation, the solution around the cells is collected and the amount of radioactively labeled PGE₂ in the solution is determined using, for example, a scintillation counter. Compounds that specifically bind to the EP₁ receptor variant displace radioactively labeled PGE_{2α} from the receptor and thereby increase radioactively labeled PGE_{2α} in the solution. A method for a whole cell radioligand binding assay using PGE₂ is described, for example, in Fujino et al., J. Biol. Chem. 275:29907-29914 (2000). As understood by one skilled in the art, a ligand such as PGE₂ also can be labeled with a non-radioactive moiety such as a fluorescent moiety.

A variety of other assays well known in the art can be used to determine specific binding of a compound to an EP₁ receptor variant in a method of the invention. Such assays include, without limitation, detecting specific binding of a labeled compound to an EP₁ receptor variant which is immobilized. For example, a compound can be conjugated to a radiolabel, fluorescent label or enzyme label such as alkaline phosphatase, horse radish peroxidase or luciferase. Labeled compound can then bind to an EP₁ receptor variant, for example an EP₁ receptor variant membrane preparation, which is immobilized, for example, on a solid support such as a latex bead. Unbound compound can be washed away, and the amount of specifically bound compound can be detected based on its label. Fluorescently labeled compound can also be bound to an EP₁ receptor variant in solution and bound complexes detected, for example, using a fluorescence polarization assay (Degterev et al., Nature Cell Bioloqy 3:173-182 (2001)). Such assays also can be performed where the EP₁ receptor variant is labeled and the compound is immobilized or in solution. One skilled in the art understands that a variety of additional means can be used to determine specific binding to an EP₁ receptor variant; as non-limiting examples, binding of a compound to a ¹⁵N-labeled EP₁ receptor variant can be detected using nuclear magnetic resonance (NMR), or specific binding can be determined using an antibody that specifically recognizes a ligand-bound EP₁ receptor variant.

High-throughput assays for determining specific binding to an EP₁ receptor variant further include, but are not limited to, scintillation proximity assays (Alouani, Methods Mol. Biol. 138:135-41 (2000)). Scintillation proximity assays involve the use of a fluomicrosphere coated with an acceptor molecule, such as an antibody, to which an antigen will bind selectively in a reversible manner. For example, a compound can be bound to a fluomicrosphere using an antibody that specifically binds to the compound, and contacted with a labeled EP₁ receptor variant. If the labeled EP₁ receptor variant specifically binds to the compound, the radiation energy from the labeled EP₁ receptor variant is absorbed by the fluomicrosphere, thereby producing light which is easily measured. Such assays can also be performed where the EP₁ receptor variant is bound to the fluomicrosphere, and the compound is labeled.

Additional assays suitable for determining specific binding of a compound to an EP₁ receptor variant in a screening method of the invention include, without limitation, UV and chemical cross-linking assays (Fancy, Curr. Opin. Chem. Biol. 4:28-33 (2000)) and biomolecular interaction analyses (Weinberger et al., Pharmacogenomics 1:395-416 (2000)). Specific binding of a compound to an EP₁ receptor variant can be determined by cross-linking these two components, if they are in contact with each other, using UV or a chemical cross-linking agent. In addition, a biomolecular interaction analysis (BIA) can detect whether two components are in contact with each other. In such an assay, one component, such as an EP₁ receptor variant (for example, a membrane preparation containing an EP₁ receptor variant) is bound to a BIA chip, and a second component such as a compound is passed over the chip. If the two components specifically bind, the contact results in an electrical signal which is readily detected.

In addition, virtual computational methods and the like can be used to identify compounds that modulate or specifically bind to an EP₁ receptor variant in a screening method of the invention. Exemplary virtual computational methodology involves virtual docking of small-molecule compounds on a virtual representation of an EP₁ receptor variant structure in order to determine or predict specific binding. See, for example, Shukur et al., supra, 1996; Lengauer et al., Current Opinions in Structural Biology 6:402-406 (1996); Choichet et al., Journal of Molecular Biology 221:327-346 (1991); Cherfils et al., Proteins 11:271-280 (1991); Palma et al., Proteins 39:372-384 (2000); Eckert et al., Cell 99:103-115 (1999); Loo et al., Med. Res. Rev. 19:307-319 (1999); and Kramer et al., J. Biol. Chem. (2000).

One type of assay that does not directly measure binding to an EP₁ receptor variant, but measures activation of a signal transduction pathway, is an assay based on melanophores, which are skin cells that provide pigmentation to an organism (Lerner, Trends Neurosci. 17:142-146 (1994)). In numerous animals, including fish, lizards and amphibians, melanophores are used, for example, for camouflage. The color of the melanophore is dependent on the intracellular position of melanin-containing organelles, termed melanosomes. Melanosomes move along a microtubule network and are clustered to give a light color or dispersed to give a dark color. The distribution of melanosomes is regulated by G protein coupled receptors and cellular signaling events, where increased concentrations of second messengers such as cyclic AMP and diacylglycerol result in melanosome dispersion and darkening of melanophores. Conversely, decreased concentrations of cyclic AMP and diacylglycerol result in melanosome aggregation and lightening of melanophores.

A melanophore-based assay can be advantageously used to identify a compound that modulates or specifically binds to an EP₁ receptor variant, due to the regulation of melanosome distribution by EP₁ receptor variant-stimulated intracellular signaling. For example, an EP₁ receptor variant can be over-expressed in genetically engineered melanophore cells, for example, frog melanophore cells. Compounds that modulate or specifically bind to the EP₁ receptor variant can stimulate or inhibit G protein coupled receptor signaling. Both stimulation or inhibition of signaling can be determined since the system can be used to detect both aggregation of melanosomes and lightening of cells, and dispersion of melanosomes and darkening of cells. Thus, the color of the cells, determined by the level of melanin in the cells, is an indicator that can be used to identify a compound that modulates or specifically binds to an EP₁ receptor variant in a method of the invention.

In addition to the methods described above for identifying a compound that modulates or specifically binds an EP₁ receptor variant, the invention also provides related methods for identifying a compound that differentially modulates or differentially binds to an EP₁ receptor variant. It is understood that the EP₁ receptor variants, cells, compounds, indicators, conditions for contacting, and assays, described above also can be applied to methods for identifying a compound that differentially modulates or differentially binds to an EP₁ receptor variant.

Provided herein is a method for identifying a compound that differentially modulates an EP₁ receptor variant by a) contacting an isolated EP₁ receptor variant or an EP₁ receptor variant over-expressed in a genetically engineered cell with a compound; b) determining the level of an indicator which correlates with modulation of an EP₁ receptor variant; c) contacting a second receptor with the compound; d) determining the level of a corresponding indicator after contacting of the compound to the second receptor; and e) comparing the level of the indicator from step (b) with the level of the corresponding indicator from step (d), where a different level of the indicator from step (b) compared to the level of the corresponding indicator from step (d) indicates that the compound is a compound that differentially modulates the EP₁ receptor variant, and where the EP₁ receptor variant contains an amino acid sequence having at least 50% amino acid identity with SEQ ID NO: 8 and containing the amino acid sequence of SEQ ID NO: 4 or 6, or a conservative variant thereof.

As described above, an indicator is a detectable substance which is altered qualitatively or quantitatively in response to modulation of an EP₁ receptor variant. A “corresponding indicator” is an indicator that can be compared to the indicator which correlates with modulation of the EP₁ receptor variant in step (b). For example, a corresponding indicator can be the same indicator as the indicator which correlates with modulation of the EP₁ receptor variant in step (b). In addition, for example, a corresponding indicator can be a different indicator as the indicator which correlates with modulation of the EP₁ receptor variant in step (b) so long as the corresponding indicator can be compared to the indicator which correlates with modulation of the EP₁ receptor variant in step (b). As a non-limiting example, the indicator in step (b) can be calcium, and the corresponding indicator can be a substance whose amount is directly correlated with calcium level, such as a signal transduction molecule. As a further non-limiting example, the indicator in step (b) and corresponding indicator in step (d) can be related molecules, such as two different fluorophores. In one embodiment, the level of the indicator which correlates with modulation of the EP₁ receptor variant in step (b) is greater than the level of the corresponding indicator from step (d). In another embodiment, the level of the indicator which correlates with modulation of the EP₁ receptor variant in step (b) is less than the level of the corresponding indicator from step (d).

The invention also provides a method for identifying a compound that differentially binds to an EP₁ receptor variant by a) contacting an isolated EP₁ receptor variant or an EP₁ receptor variant over-expressed in a genetically engineered cell with a compound; b) determining specific binding of the compound to the EP₁ receptor variant; c) contacting a second receptor with the compound; d) determining specific binding of the compound to the second receptor; and e) comparing the level of specific binding from step (b) with the level of specific binding from step (d), where a different level of specific binding from step (b) compared to the level of specific binding from step (d) indicates that the compound is a compound that differentially binds to the EP₁ receptor variant, and where the EP₁ receptor variant contains an amino acid sequence having at least 50% amino acid identity with SEQ ID NO: 8 and containing the amino acid sequence of SEQ ID NO: 4 or 6, or a conservative variant thereof. In one embodiment, the different level of specific binding is an increased level of binding. In another embodiment, the different level of specific binding is a decreased level of binding.

As set forth above in regard to methods for identifying a compound that modulates or specifically binds an EP₁ receptor variant, the EP₁ receptor variant can be any of a variety of EP₁ receptor variants such as an isolated polypeptide containing an amino acid sequence having at least 50% amino acid identity with SEQ ID NO: 8 and containing the amino acid sequence of SEQ ID NO: 4 or 6, or a conservative variant thereof; or an isolated polypeptide containing the amino acid sequence of SEQ ID NO: 2, or a conservative variant thereof. In addition, the EP₁ receptor variant can be over-expressed in a genetically engineered cell. For example, the EP₁ receptor variant can be exogenously over-expressed in a genetically engineered cell.

In the methods of the invention for identifying a compound that differentially modulates or differentially binds an EP₁ receptor variant, the second receptor can be any receptor of interest. For example, the second receptor can be a G-protein coupled receptor such as, without limitation, any other-EP₁ receptor such as a different EP₁ receptor variant or a wild-type EP₁ receptor. In particular embodiments, the second receptor is a wild-type EP₁ receptor containing the amino acid sequence SEQ ID NO: 8, or a functional fragment thereof.

The second receptor can be, for example, expressed in a cell endogenously or exogenously or can be an isolated polypeptide.

It is understood that the methods of the invention can be practiced where the EP₁ receptor variant and second receptor are expressed, for example, in different cells. In addition, the methods of the invention can be practiced where the EP₁ receptor variant and second-receptor are expressed in the same cell, for example, where the EP₁ receptor variant does not have identical binding and signal transduction effects as the co-expressed second receptor.

The invention also provides an isolated nucleic acid-molecule having a nucleotide sequence that encodes a polypeptide containing an amino acid sequence having at least 50% amino acid identity with SEQ ID NO: 8 and containing the amino acid sequence of SEQ ID NO: 4 or 6, or a conservative variant thereof. The invention also provides an isolated nucleic acid molecule having a nucleotide sequence that encodes a polypeptide containing an amino acid sequence having at least 80% or at least 90% amino acid identity with SEQ ID NO: 8 and containing the amino acid sequence of SEQ ID NO: 4 or 6, or a conservative variant thereof. The invention further provides an isolated nucleic acid molecule containing a nucleotide sequence that encodes the amino acid sequence of SEQ ID NO: 2, or a conservative variant thereof. The invention also provides an isolated nucleic acid molecule containing a nucleotide sequence that encodes SEQ ID NO: 2, such as the nucleotide sequence of SEQ ID NO: 1. The invention further provides an isolated nucleic acid molecule that is distinct from EST sequence BE256373.

Isolated nucleic acid molecules include DNA and RNA molecules as well as both sense or complementary anti-sense strands. It is understood that an isolated nucleic acid molecule of the invention can be a double-stranded or single-stranded molecule, an RNA or DNA molecule, and can optionally include non-coding sequence. DNA molecules of the invention include cDNA sequences as well as wholly or partially chemically synthesized DNA sequences.

The nucleic acid molecules of the invention optionally include heterologous nucleic-acid sequences that are not part of the EP₁ receptor variant-encoding sequences in nature. Such a heterologous nucleic acid sequence can be optionally separated from the EP₁ receptor variant-encoding sequence by an encoded cleavage site that facilitates removal of non-EP₁ receptor variant polypeptide sequences from the expressed fusion protein. Heterologous nucleic acid sequences include, without limitation, sequences encoding poly-histidine sequences, FLAG tags and other epitopes, and glutathione-S-transferase, thioredoxin, and maltose binding protein domains or other domains or sequences that facilitate purification or detection of the fusion protein containing an EP₁ receptor variant of the invention.

The location of exons from the human EP₁ receptor genomic clone AC008569.7 that are present in alternatively spliced human EP₁ receptor variant EP₁ VAR-1 as determined using BLAST searches indicate that EP₁ receptor variant VAR-1 alternatively spliced sequence corresponds to human genomic clone AC008569.7 at a range from +207985 to +207436.

The invention further provides a vector containing a nucleic acid molecule having a nucleotide sequence that encodes a polypeptide containing an amino acid sequence having at least 50% amino acid identity with SEQ ID NO: 8 and containing the amino acid sequence of SEQ ID NO: 4 or 6, or a conservative variant thereof. The invention also provides a vector containing a nucleic acid molecule containing a nucleotide sequence that encodes the amino acid sequence of SEQ ID NO: 2, or a conservative variant thereof. In addition, the invention also provides a vector containing a nucleic acid molecule containing or consisting of SEQ ID NO: 2. For example, such a vector can contain a nucleic acid molecule having the nucleotide sequence of SEQ ID NO: 1. The invention further provides a host cell including a vector which contains a nucleic acid molecule of the invention.

Vectors are useful, for example, for subcloning and amplifying a nucleic acid molecule encoding a polypeptide of the invention and for recombinantly expressing the encoded EP variant receptor or other polypeptide. Vectors of the invention include, without limitation, viral vectors such as a bacteriophage, baculovirus and retrovirus vectors; cosmids or plasmids; and, particularly for cloning large nucleic acid molecules, bacterial artificial chromosome vectors (BACs) and yeast artificial chromosome vectors (YACs). Such vectors are commercially available, and their uses are well known in the art. Vectors further encompass expression vectors such as those discussed herein above.

As understood by one skilled in the art, a nucleic acid molecule of the invention can contain nucleotide sequence in addition to nucleotide sequence encoding an EP₁ variant polypeptide of the invention. For example, a nucleic acid molecule of the invention can include one or more additional heterologous sequences such as nucleotide sequences encoding restriction enzyme sites or epitope tags. As non-limiting examples, nucleic acid molecules of the invention can be used in hybridization reactions such as Southern and Northern blots, to encode polypeptide sequence in recombinant cloning methods, or as primers in polymerase chain reactions.

The invention also provides an isolated nucleic acid molecule having a nucleotide sequence that encodes a polypeptide containing or consisting of substantially the same amino acid sequence as SEQ ID NO: 2 For example, the invention provides an isolated nucleic acid molecule having the nucleotide sequence of SEQ ID NO: 1.

The invention further provides a method for preventing or reducing the severity of a disease associated with an EP₁ receptor or an EP₁ receptor variant in a subject by introducing into the subject a compound that modulates or specifically binds to an EP₁ receptor variant or another compound identified by a method of the invention. The invention also provides a method for regulating pain in a subject by introducing into the subject a compound that modulates or specifically binds to an EP₁ receptor variant or another compound identified by a method of the invention. In addition, the invention provides a method for preventing or reducing the severity of a cardiovascular disease in a subject by introducing into the subject a compound that modulates or specifically binds to an EP₁ receptor variant or another compound identified by a method of the invention.

As used herein, a “disease associated with an EP₁ receptor or EP₁ receptor variant” means any disease or condition in which modulation of the activity of the wild-type EP₁ receptor or an EP₁ receptor variant can be beneficial. It is understood that the underlying cause of the disease may or may not be due to an abnormality in expression or activity of a wild-type EP₁ receptor or EP₁ receptor variant.

A disease associated with an EP₁ receptor or EP₁ receptor variant can be, without limitation, pain, a cardiovascular disorder or an ocular disorder such as glaucoma or ocular hypertension. Additional diseases or conditions associated with an EP₁ receptor or an EP₁ receptor variant can include, without limitation, diseases involving the kidney, central nervous system, gastrointestinal tract, blood pressure, or cancer. As non-limiting examples, such a compound can be used in humans for prophylatic treatment of a cardiovascular disorder, disorder of the central nervous system, or gastrointestinal disorder. Such a compound also can be used to prevent or treat pain such as headache; muscle ache; pain caused by any of a variety of inflammatory or degenerative joint diseases; and ocular, nasal or cutaneous itch.

A compound identified by the methods of the invention can be used, without limitation, to prevent or reduce the severity of glaucoma. Glaucoma, the second most common cause of blindness in the United States, affects about two million Americans, but roughly half are unaware of it. This group of disorders is characterized by progressive damage to the eye at least partly due to intraocular pressure. Normal intraocular pressure (IOP) ranges between 11 and 21 mm Hg; however, this level may not necessarily be healthy for all people. Some people with normal pressure develop optic nerve injury (normal- or low-pressure glaucoma). In contrast, many people have pressure greater than 21 mm Hg without any optic nerve injury (ocular hypertension). Of those with ocular hypertension, only about 1% per year will develop glaucoma.

Glaucoma can be described according to the mechanism of outflow obstruction as either open-angle or closed-angle (angle-closure) glaucoma. Alternatively, classification can be based on etiology as primary or secondary. The primary (conventional) outflow system of the eye is located in the anterior chamber angle and accounts for 83 to 96% of aqueous outflow in human eyes under normal circumstances. The primary outflow system refers to aqueous outflow through the trabecular meshwork, canal of Schlemm, intrascleral channels, and episcleral and conjunctival veins. In open-angle glaucoma with elevated intraocular pressure, pressure elevation occurs because outflow is inadequate despite an angle that appears open and relatively normal on gonioscopic examination. In closed-angle glaucoma, elevated intraocular pressure occurs when normal drainage of aqueous fluid from the eye is sufficiently prevented by a physical obstruction of the peripheral iris. The secondary (alternative) aqueous outflow pathways (known as the unconventional or uveoscleral aqueous outflow system) account for 5 to 15% of the total aqueous outflow. The secondary aqueous outflow pathway refers to aqueous exiting the eye through the anterior face of the ciliary body and percolating through the ciliary muscles to the suprachoroidal space (i.e., between the choroid and sclera), where it eventually exits the eye via scleral channels. It is understood that compounds that modulate or specifically bind to an EP₁ receptor variant or that are otherwise identified according to a method of the invention can be used to treat any of a variety of forms of glaucoma including, but not limited to, normal- or low-pressure glaucoma, glaucoma with elevated intraocular pressure, primary glaucoma and secondary glaucoma.

Furthermore, a compound that modulates or specifically binds to an EP₁ receptor variant or which is otherwise identified by a method of the invention can be used alone or in combination with one or more different compounds or other therapeutics or procedures for treatment of glaucoma. Compounds that are currently used in the treatment of glaucoma include, but are not limited to, topical-blockers such as timolol, levobunolol, carteolol, metipranolol and betaxolol; topical nonselective adrenergic agonists such as epinephrine and dipivefrin; adrenergic agonists such as apraclonidine and brimonidine; topical cholinergic agonists such as pilocarpine and phospholine; oral carbonic anhydrase inhibitors such as acetazolamide and methazolamide; topical carbonic anhydrase inhibitors such as dorzolamide; and topical prostaglandin analogs such as latanoprost, unoprostone, and travoprost.

Other ocular conditions that can be prevented or treated with a compound that modulates or differentially modulates an EP₁ receptor variant by a method of the invention include, without limitation, diabetic retinopathy; macular edema such as that associated with diabetes; conditions of retinal degeneration, for example, macular degeneration such as age-related macular degeneration (ARMD) and retinitis pigmentosa; retinal dystrophies; inflammatory disorders of the retina; vascular occlusive conditions of the retina such as retinal vein occlusions or branch or central retinal artery occlusions; retinopathy of prematurity; retinopathy associated with blood disorders such as sickle cell anemia; elevated intraocular pressure; ocular itch; damage following retinal detachment; damage or insult due to vitrectomy, retinal or other surgery; and other retinal damage including therapeutic damage such as that resulting from laser treatment of the retina, for example, pan-retinal photocoagulation for diabetic retinopathy or photodynamic therapy of the retina, for example, for age-related macular degeneration. Ocular conditions that can be prevented or treated with a compound that modulates or differentially modulates an EP₁ receptor variant by a method of the invention further include, without limitation, genetic and acquired optic neuropathies such as optic neuropathies characterized primarily by loss of central vision, for example, Leber's hereditary optic neuropathy (LHON), autosomal dominant optic atrophy (Kjer disease) and other optic neuropathies such as those involving mitochondrial defects, aberrant dynamin-related proteins or inappropriate apoptosis; and optic neuritis such as that associated with multiple sclerosis, retinal vein occlusions or photodynamic or laser therapy. See, for example, Carelli et al., Neurochem. Intl. 40:573-584 (2002); and Olichon et al., J. Biol. Chem. 278:7743-7746 (2003).

A compound that modulates or differentially modulates an EP₁ receptor variant or another compound identified by a method of the invention also can be useful for preventing or treating pain. The term pain, as used herein, includes, without limitation, inflammatory pain, headache pain, muscle pain, visceral pain, neuropathic pain, and referred pain. Pain can be continuous or intermittent, of short duration such as acute pain, or of long duration such as chronic pain. Chronic pain is distinguished from acute pain, which is immediate, generally high threshold, pain brought about by injury such as a cut, crush, burn, or by chemical stimulation such as that experienced upon exposure to capsaicin, the active ingredient in chili peppers.

The methods of the invention further can be used, without limitation, to treat chronic or other headache pain such as pain associated with cluster headaches, tension headaches or chronic daily headaches; muscle pain including, but not limited to, that associated with back or other spasm; inflammatory pain or other symptoms resulting, for example, from spondylitis or arthritis such as rheumatoid arthritis, gouty arthritis, or osteoarthritis; gout; bursitis; painful menstruation and fever. In addition, the methods of the invention can be used, for example, to treat pain associated with injury, surgery, dental procedures, dysmenorrhea, labor and other pain associated with the female reproductive system, and systemic illness such as, without limitation, cancer. It is understood that these and other conditions which may respond to NSAIDs can be prevented or treated using a compound that modulates or differentially modulates an EP₁ receptor variant disclosed herein.

As described above, a compound that modulates or differentially modulates an EP₁ receptor variant or another compound identified by a method of the invention also can be useful for preventing or treating an immune disease, for example, without limitation, uveitis. In addition, such a compound can be used to prevent or treat a disease associated with allergy, such as, without limitation, allergic conjunctivitis.

A compound that modulates or differentially modulates an EP₁ receptor variant or another compound identified by a method of the invention also can be useful for preventing or treating a cardiovascular disorder. Such cardiovascular diseases include, but are not limited to, atherosclerosis; thrombosis; restenosis; vasculitis including autoimmune and viral vasculitis such as polyarteritis nodosa, Churg-Strass syndrome, Takayasu's arteritis, Kawasaki Disease and Rickettsial vasculitis; atherosclerotic aneurisms; myocardial hypertrophy; congenital heart diseases (CHD); ischemic heart disease and anginas; acquired valvular/endocardial diseases; primary myocardial diseases including myocarditis; arrhythmias; and cardiac tumors.

In the methods of the invention for preventing or reducing the severity of pain or glaucoma or another disease associated with an EP₁ receptor or EP₁ receptor variant, a compound can optionally be formulated together with a pharmaceutically acceptable carrier for delivery to the subject to be treated. Suitable pharmaceutically acceptable carriers are well known in the art and include, for example, aqueous or organic solvents such as physiologically buffered saline, glycols, glycerol, oils or injectable organic esters. A pharmaceutically acceptable carrier can also contain a physiologically acceptable agent that acts, for example, to stabilize or increase solubility of a pharmaceutical composition. Such a physiologically acceptable agent can be, for example, a carbohydrate such as glucose, sucrose or dextrans; an antioxidant such as ascorbic acid or glutathione; a chelating agent; a low molecular weight polypeptide; or another stabilizer or excipient. Pharmaceutically acceptable carriers including solvents, stabilizers, solubilizers and preservatives, are well known in the art as described, for example, in Martin, Remington's Pharm. Sci., 15th Ed. (Mack Publ. Co., Easton, 1975).

Ophthalmic compositions can be useful in the methods of the invention for preventing or alleviating an ocular condition. An ophthalmic composition contains an ophthalmically acceptable carrier, which is any carrier that has substantially no long term or permanent detrimental effect on the eye to which it is administered. Examples of ophthalmically acceptable carriers include, without limitation, water, such as distilled or deionized water; saline; and other aqueous media.

Topical ophthalmic compositions useful for alleviating an ocular condition include, without limitation, ocular drops, ocular ointments, ocular gels and ocular creams. Such ophthalmic compositions are easy to apply and deliver the active compound effectively.

A preservative can be included, if desired, in an ophthalmic composition useful in a method of the invention. Such a preservative can be, without limitation, benzalkonium chloride, chlorobutanol, purite, thimerosal, phenylmercuric acetate, or phenylmercuric nitrate. Vehicles useful in a topical ophthalmic composition include, yet are not limited to, polyvinyl alcohol, povidone, hydroxypropyl methyl cellulose, poloxamers, carboxymethyl cellulose, hydroxyethyl cellulose and purified water.

A tonicity adjustor also can be included, if desired, in an ophthalmic composition administered to alleviate an ocular condition without concomitant sedation according to a method of the invention. Such a tonicity adjustor can be, without limitation, a salt such as sodium chloride, potassium chloride, mannitol or glycerin, or another pharmaceutically or ophthalmically acceptable tonicity adjustor.

Various buffers and means for adjusting pH can be used to prepare an ophthalmic composition useful in the invention, provided that the resulting preparation is ophthalmically acceptable. Such buffers include, but are not limited to, acetate buffers, citrate buffers, phosphate buffers and borate buffers. It is understood that acids or bases can be used to adjust the pH of the composition as needed. Ophthalmically acceptable antioxidants useful in preparing an ophthalmic composition include, yet are not limited to, sodium metabisulfite, sodium thiosulfate, acetylcysteine, butylated hydroxyanisole and butylated hydroxytoluene.

Those skilled in the art can formulate a compound that modulates, differentially modulates, specifically binds, or differentially binds an EP₁ receptor variant to ensure proper compound distribution and bioavailablility in vivo. For example, some regions of the eye can be inaccessible to some systemically administered drugs, and as a result topical drug delivery can be used. Polymers can be added to ophthalmic solutions to increase bioavailability (Ludwig and Ootenhgm, S.T.P. Pharm. Sci., 2:81-87 (1992)). In addition, colloidal systems such as, without limitation, liposomes, microparticles or nanoparticules can be used to increase penetration of a compound into the eye. Ocular drug absorption also can be enhanced using, for example, iontophoresis, prodrugs, and cyclodextrins.

Methods of ensuring appropriate distribution in vivo also can be provided by rechargeable or biodegradable devices, particularly where concentration gradients or continuous delivery is desired. Various slow release polymeric devices are known in the art for the controlled delivery of drugs, and include both biodegradable and non-degradable polymers and hydrogels. Polymeric device inserts can allow for accurate dosing, reduced systemic absorption and in some cases, better patient compliance resulting from a reduced frequency of administration. Those skilled in the art understand that the choice of the pharmaceutical formulation and the appropriate preparation of the compound will depend on the intended use and mode of administration.

A compound that modulates or specifically binds to an EP₁ receptor variant, or that is otherwise identified by a screening method of the invention can be administered to a subject by any effective route. Suitable routes of administration include, but are not limited to, oral, topical, intraocular, intradermal, parenteral, intranasal, intravenous, intramuscular, intraspinal, intracerebral and subcutaneous routes. The present invention also provides compounds containing an acceptable carrier such as any of the standard pharmaceutical carriers, including phosphate buffered saline solution, water and emulsions such as an oil and water emulsion, and various types of wetting agents.

A method of the invention is practiced by peripherally administering to a subject an effective amount of a compound that modulates or differentially modulates an EP₁ receptor variant or another compound identified by a method of the invention. As used herein in reference to such a compound, the term “peripherally administering” or “peripheral administration” means introducing the compound into a subject outside of the central nervous system. Thus, peripheral administration encompasses any route of administration other than direct administration to the spine or brain.

An effective amount of a compound of the invention can be peripherally administered to a subject by any of a variety of means depending, for example, on the type of condition to be alleviated, the pharmaceutical formulation, and the history, risk factors and symptoms of the subject. Routes of peripheral administration suitable for the methods of the invention include both systemic and local administration. As non-limiting examples, an effective amount of a compound of the invention can be administered orally; sublingually; parenterally; by subcutaneous pump; by dermal patch; by intravenous, intra-articular, subcutaneous or intramuscular injection; by topical drops, creams, gels or ointments; as an implanted or injected extended release formulation; or by subcutaneous minipump or other implanted device, and by inhalation by aerosol and similar devices.

One skilled in the art understands that peripheral administration can be local or systemic. Local administration results in significantly more of a compound of the invention being delivered to and about the site of local administration than to regions distal to the site of administration. Systemic administration results in delivery of a compound of the invention essentially throughout at least the entire peripheral system of the subject.

Routes of peripheral administration useful in the methods of the invention encompass, without limitation, oral administration, sublingual administration, topical administration, intravenous or other injection, and implanted minipumps or other extended release devices or formulations. A compound of the invention can be peripherally administered, without limitation, orally in any acceptable form such as in a tablet, pill, capsule, powder, liquid, suspension, emulsion or the like; an aerosol; as a suppository; by intravenous, intraperitoneal, intramuscular, subcutaneous or parenteral injection; by transdermal diffusion or electrophoresis; topically in any acceptable form such as in drops, creams, gels or ointments; and by minipump or other implanted extended release device or formulation. A compound of the invention optionally can be packaged in unit dosage form suitable for single administration of precise dosages, or in sustained release dosage form for continuous controlled administration.

Chronic pain and other chronic conditions such as, without limitation, chronic neurological conditions can be alleviated using any of a variety of forms of repeated or continuous administration as necessary. In the methods of the invention for alleviating chronic pain or another chronic condition, means for repeated or continuous peripheral administration include, without limitation, repeated oral or topical administration, and administration via subcutaneous minipump. As non-limiting examples, a method of the invention can be practiced by continuous intravenous administration via implanted infusion minipump, or using an extended release formulation.

It is understood that slow-release formulations can be useful in the methods of the invention for alleviating chronic pain or other chronic conditions such as, without limitation, a chronic neurodegenerative conditions. It is further understood that the frequency and duration of dosing will be dependent, in part, on the alleviation desired and the half-life of the compound of the invention and that a variety of routes of administration are useful for delivering slow-release formulations, as detailed herein above.

A compound of the invention can be peripherally administered to a subject to alleviate an ocular condition by any of a variety of means depending, in part, on the characteristics of the compound to be administered and the history, risk factors and symptoms of the subject. Peripheral routes of administration suitable for alleviating an ocular condition in a method of the invention include both systemic and local administration. In particular embodiments, a pharmaceutical composition containing a compound of the invention is administered topically, or by local injection, or is released from an intraocular or periocular implant.

Systemic and local routes of administration useful in alleviating an ocular condition according to a method of the invention encompass, without limitation, oral gavage; intravenous injection; intraperitoneal injection; intramuscular injection; subcutaneous injection; transdermal diffusion and electrophoresis; topical eye drops and ointments; periocular and intraocular injection including subconjunctival injection; extended release delivery devices such as locally implanted extended release devices; and intraocular and periocular implants including bioerodible and reservoir-based implants.

In one embodiment, a method of the invention for alleviating an ocular condition is practiced by administering an ophthalmic composition containing a compound of the invention topically to the eye. The compound can be administered, for example, in an ophthalmic solution (ocular drops). In another embodiment, an ophthalmic composition containing a compound of the invention is injected directly into the eye. In a further embodiment, an ophthalmic composition containing a compound of the invention is released from an intraocular or periocular implant such as a bioerodible or reservoir-based implant.

As indicated above, an ophthalmic composition containing a compound of the invention can be administered locally via an intraocular or periocular implant, which can be, without limitation, bioerodible or reservoir-based. An implant refers to any material that does not significantly migrate from the insertion site following implantation. An implant can be biodegradable, non-biodegradable, or composed of both biodegradable and non-biodegradable materials; a non-biodegradable implant can include, if desired, a refillable reservoir. Implants useful in a method of the invention for alleviating an ocular condition include, for example, patches, particles, sheets, plaques, microcapsules and the like, and can be of any shape and size compatible with the selected site of insertion, which can be, without limitation, the posterior chamber, anterior chamber, suprachoroid or subconjunctiva of the eye. It is understood that an implant useful in the invention generally releases the implanted pharmaceutical composition at an effective dosage to the eye of the subject over an extended period of time. A variety of ocular implants and extended release formulations suitable for ocular release are well known in the art, as described, for example, in U.S. Pat. Nos. 5,869,079 and 5,443,505.

An effective dose of a compound for use in a method of the invention can be determined, for example, by extrapolation from the concentration required in a an EP₁ receptor or EP₁ receptor variant binding or activity assay such as one of the assays disclosed herein above. An effective dose of a compound for the treatment of a disease associated with an EP₁ receptor or EP₁ receptor variant also can be determined from appropriate animal models, such as transgenic mice. As non-limiting examples, animal models for pathologies such as cardiovascular disease and ocular diseases are well-known in the art. An effective dose for preventing or reducing the severity of a disease is a dose that results in either partial or complete alleviation of at least one symptom of the disease. The appropriate dose of a compound for treatment of a human subject can be determined by those skilled in the art, and is dependent, for example, on the particular disease being treated, nature and bioactivity of the particular compound, the desired route of administration, the gender, age and health of the individual, and the number of doses and duration of treatment.

All journal article, reference and patent citations provided herein, including referenced sequence accession numbers of nucleotide and amino acid sequences contained in various databases, in parentheses or otherwise, whether previously stated or not, are incorporated herein by reference in their entirety.

It is understood that modifications which do not substantially affect the activity of the various embodiments of this invention are also included within the definition of the invention provided herein. Accordingly, the following examples are intended to illustrate but not limit the present invention.

### EXAMPLE I

### Identification of an Alternatively Spliced EP₁ Receptor Variant

This example shows the molecular cloning of the alternatively spliced EP₁ receptor variant EP₁ VAR-1 and its expression in cell culture.

Total RNA derived from human heart, brain, lung, spleen, small intestine, skeletal muscle, kidney and liver tissue were purchased from Clontech. Total RNA was isolated from human eyes (NDR1; Philadelphia, Pa.) and human ocular tissues (ciliary smooth muscles, trabecular meshwork, ODM-2) using a Qiagen total RNA isolation kit, according to the manufacturer's instructions. The ODM-2 cell line is derived from human non-pigmented ciliary epithelial cells (Escribano et al., J. Cell. Physiol. 160:511-521 (1994)). Using 5 μg of human total RNA, first strand cDNA was synthesized using SuperScript II RNase H reverse transcriptase (Life Technologies; Carlsbad, Calif.). Reactions (20 μl) containing 5 μg of RNA, 250 ng of oligo (dT), and 100 units of reverse transcriptase were incubated at 42° C. for 1 hour and terminated by incubation at 100° C. for 3 minutes. The PCR buffer contained 10 mM Tris-HCl, pH 8.3, 50 mM KCl, 2 mM MgCl, 2.5 units AmpliTaq DNA polymerase, 0.2 μM upstream and downstream primers, in a final volume of 50 μl. After an initial incubation for 5 minutes at 94° C., samples were subjected to 30 cycles of 30 seconds at 95° C., 30 seconds at 58° C., and 30 seconds at 72° C. in a PE 9700 thermal cycler. The primers used for the detection of the alternatively spliced EP₁ receptor variant EP₁ VAR-1 were as follows:

(SEQ ID NO: 9)

Human EP1 Forward: GGTATCATGGTGGTGTCGTG
and

(SEQ ID NO: 10)

Human EP1 Reverse: CCTGGCGCAGTAGGATGTA

The PCR products were isolated from a 1.5% lower melting agarose gel, and subcloned into the TOPO PCRII vector (Invitrogen; Carlsbad, Calif.). Nucleotide sequencing of the vectors was performed by Sequetech (Mountain View, Calif.).

Full length cDNA for the EP₁ receptor variant EP₁ VAR-1 was isolated and subcloned into TOPO pcDNA3.1 PCR cloning vector (Invitrogen; Carlsbad, Calif.) or pCEP4 expression vector (Invitrogen) to create Alt EP/pcDNA3.1 plasmids or Alt FP/pCEP4 plasmids. Alt EP/pcDNA3.1 plasmids were used for transient transfection, and Alt EP/pCEP4 plasmids were used for stable transfection. Full length Gα₁₆ cDNA was subcloned into the pcDNA3.1 vector. The plasmids were sequenced by Sequetech.

HEK 293/EBNA cells were obtained from the American Type Culture Collection (ATCC). HEK 293/EBNA cells were routinely maintained in DMEM with 10% fetal bovine serum, 1% glutamine, 0.5% penicillin/streptomycin. Cells were kept in humidified 5% CO₂, 95% air at 37° C. For stable transfection, Alt EP/pCEP4 plasmids were transfected into HEK 293/EBNA cells using Fugene 6 (Roche Diagnostics Corp., Inc.; Indianapolis, Ind.), according to the manufacture's instructions, and then 200 mg/ml hygromycin was used to select cell clones that stably expressed the plasmid.

### EXAMPLE II

### Tissue Distribution of Alternatively Spliced EP₁ Receptor Variant EP₁ VAR-1

This example shows the tissue distribution of alternatively spliced EP₁ receptor variant EP₁ VAR-1 mRNA using multiple tissue Northern blots.

Total RNA was isolated from human ciliary body using RNeasy Kit (Qiagen, Inc), according to the manufacturer's instruction. RNA concentrations were determined by UV Spectrophotometry (Beckman DU640) at A 260 nM, and stored at −80° C. Total RNA (10 μg) was denatured at 65° C. in RNA loading buffer (Ambion, Inc.) for 15 minutes and separated on 1.2% agarose gels containing 0.66 M formaldehyde. The RNAs were transferred to a nitrocellulose membrane. A human MutipleTissue Northern Blot was purchased from Clontech. Human 292 bp Human Alt EP₁ (+956 bp to +1248 bp; Human Alt EP1 sequence) specific DNA fragment was radiolabled using α-³²P dATP and Klenow (Ambion, Inc.). The blots were hybridized with gene specific probes in 50% formamide, 4×SSC, 1 Denhardt's solution, 50 mM sodium phosphate, pH 7.0, 1% SDS, 50 μg/ml yeast tRNA, and 0.5 mg/ml sodium pyrophosphate at 42° C. overnight. Blots were washed with 2×SSC and 0.1% SDS twice at 42° C. and 0.1×SSC and 0.1% SDS twice at 42° C. The hybridized blots were exposed to phosphor screens, and the exposed screens were analyzed in a PhosphorImager (Molecular Dynamics, Inc).

### EXAMPLE III

### Screening Assays Using EP₁ Receptor Variants

This example describes a FLIPR and luciferase assay for screening compounds against EP₁ receptor variants.

HEK 293/EBNA cells transiently or stably expressing Alt EP/pcDNA3.1 plasmids were seeded at a density of 5×10³ cells per well in Biocoat® Poly-D-lysine-coated black-wall, clear-bottom 96-well plates (Becton-Dickinson; Franklin Lakes, N.J.) and allowed to attach overnight. At 48 hours after transfection, the cells were washed two times with HBSS-HEPES buffer (Hanks Balanced Salt Solution without bicarbonate and phenol red, 20 mM HEPES, pH 7.4) using a Lab Systems Cellwash plate washer. After 45 minutes of dye-loading in the dark, using the calcium-sensitive dye Fluo-4 AM at a final concentration of 2 mM, the plates were washed four times with HBSS-HEPES buffer to remove excess dye leaving 100 μl in each well. Plates were re-equilibrated to 37° C. for a few minutes. The cells were excited with an Argon laser at 488 nm, and emission was measured through a 510-570 nm bandwidth emission filter (FLIPR1; Molecular Devices; Sunnyvale, Calif.). Compound solution was added in a 50 μl volume to each well to give the desired final concentration. The peak increase in fluorescence intensity was recorded for each well. To generate concentration-response curves, compounds were tested in duplicate in a concentration range between 10⁻¹¹ and 10⁻⁵ M. The duplicate values were averaged.

CRE-luciferase reporter plasmids purchased from Invitrogen were used for detecting cAMP accumulation in G_{α}_{^{s}} coupled receptors. pGL3-N-960 plasmids containing human Nur77 promoter (Uemura et al., J. Biol. Chem. 270:5427-5433 (1995)) and pGL3-CTGF-LUC plasmids containing human CTGF promoter were used for detecting calcium, PKC, and MAP kinase pathways associated with G_{α}_{^{q}} coupled receptors. For the pGL3-CTGF-LUC plasmid, a DNA fragment containing the CTGF promoter region from −2047 to +65 (Fu et al., J. Biol. Chem 276:45888-45894 (2001)) was cloned from human genomic DNA (Clontech). The fragment was subcloned into a pGL3 luciferase expression vector (Promega Inc.) creating the pGL3-CTGF-LUC plasmid.

Luciferase reporter plasmids were transfected into HEK 293/EBNA cells transiently or stably expressing EP₁ receptor variants using Fugene 6, according to the manufacturer's instructions. In brief, the cells were plated in 24 well plates overnight, and then the 24 well plate cells were washed twice and resuspended in 1 ml of DMEM. The cell suspension was mixed with 0.2 μg of plasmid DNA in 100 μl of DMEM containing 0.6 μl Fugene 6 solution and added into each well. Plates were cultured for 24 hours at 37° C. before compounds were added to the cultures at concentrations ranging from 10⁻¹¹ to 10⁻⁶ M. Cells were harvested 6 hours later and lysed in 100 μl of 25 mM Tris-phosphate buffer (pH 7.5) containing 1% Triton X-100. Soluble extracts (20 μl) were assayed for luciferase activity as described below.

The luciferase assay was performed with a Promega assay kit (Promega, Inc.; Madison, Wis.) at room temperature using an Autolumat LB 953 (Berthold; Bad Wildbad, Germany). Luciferase content was measured by calculating the light emitted during the initial 10 seconds of the reaction. Relative luciferase activity was expressed as fold values of ratio compared to control. Experiments were independently repeated at least 3 times.

All journal article, reference and patent citations provided herein, including referenced sequence accession numbers of nucleotide and amino acid sequences contained in various databases, in parentheses or otherwise, whether previously stated or not, are incorporated herein by reference in their entirety.

Although the invention has been described with reference to the disclosed embodiments, those skilled in the art will readily appreciate that the specific experiments detailed are only illustrative of the invention. It should be understood that various modifications can be made without departing from the spirit of the invention.

1. An isolated polypeptide, comprising
a) an amino acid sequence having at least 50% amino acid identity with SEQ ID NO: 8, and
b) an amino acid sequence of SEQ ID NO: 4 or 6; or a conservative variant thereof.

2. The isolated polypeptide of claim 1, wherein said polypeptide comprises an amino acid sequence having at least 80% amino acid identity with SEQ ID NO: 8.

3. The isolated polypeptide of claim 1, wherein said polypeptide comprises an amino acid sequence, having at least 90% amino acid identity with SEQ ID NO: 8.

4. An isolated polypeptide, comprising SEQ ID NO: 2, or a conservative variant thereof.

5. The isolated polypeptide of claim 4, wherein said polypeptide comprises SEQ ID NO: 2.

6. The isolated polypeptide of claim 5, wherein said polypeptide consists of SEQ ID NO: 2.

7. A cell, comprising the exogenously expressed polypeptide of claim 1, 2 or 4.

8. A method for identifying a compound that modulates an EP₁ receptor variant, comprising:
a) contacting said EP₁ receptor variant with a compound, wherein said EP₁ receptor variant is an isolated EP₁ receptor variant or an EP₁ receptor variant over-expressed in a genetically engineered cell, and
b) determining the level of an indicator which correlates with modulation of the EP₁ receptor variant, wherein an alteration in the level of said indicator as compared to a control level indicates that said compound is a compound that modulates the EP₁ receptor variant,
wherein said EP₁ receptor variant is the polypeptide of claim 1.

9. The method of claim 8, wherein said alteration is an increase in the level of said indicator.

10. The method of claim 8, wherein said alteration is a decrease in the level of said indicator.

11. The method of claim 8, wherein said EP₁ receptor variant is a polypeptide comprising an amino acid sequence having at least 80% amino acid identity with SEQ ID NO: 8.

12. The method of claim 8, wherein said EP₁ receptor variant is a polypeptide comprising SEQ ID NO: 2, or a conservative variant thereof.

13. The method of claim 8, wherein said EP₁ receptor variant is an isolated EP₁ receptor variant polypeptide.

14. The method of claim 8, wherein said EP₁ receptor variant is an EP₁ receptor variant over-expressed in a genetically engineered cell.

15. The method of claim 14, wherein said EP₁ receptor variant is exogenously expressed.

16. The method of claim 8, wherein said indicator is calcium.

17. The method of claim 8, wherein said compound is a polypeptide.

18. The method of claim 8, wherein said compound is a small molecule.

19. A method for identifying a compound that specifically binds to an EP₁ receptor variant, comprising:
a) contacting said EP₁ receptor variant with a compound, wherein said EP₁ receptor variant is an isolated EP₁ receptor variant or an EP₁ receptor variant over-expressed in a genetically engineered cell, and
b) determining specific binding of said compound to said EP₁ receptor variant,
wherein said EP₁ receptor variant is the polypeptide of claim 1.

20. The method of claim 19, wherein said EP₁ receptor variant is a polypeptide comprising an amino acid sequence having at least 80% amino acid identity with SEQ ID NO: 8.

21. The method of claim 19, wherein said EP₁ receptor variant is a polypeptide comprising SEQ ID NO: 2, or a conservative variant thereof.

22. The method of claim 19, wherein said EP₁ receptor variant is an isolated EP₁ receptor polypeptide.

23. The method of claim 19, wherein said EP₁ receptor is an EP₁ receptor variant over-expressed in a genetically engineered cell.

24. The method of claim 23, wherein said EP₁ receptor variant is exogenously expressed.

25. The method of claim 19, wherein said contacting occurs in vitro.

26. The method of claim 19, wherein said compound is a polypeptide.

27. The method of claim 19, wherein said compound is a small molecule.

28. A method for identifying a compound that differentially modulates an EP₁ receptor variant, comprising:
a) contacting said EP₁ receptor variant with a compound, wherein said EP₁ receptor variant is an isolated EP₁ receptor variant or an EP₁ receptor variant over-expressed in a genetically engineered cell;
b) determining the level of an indicator which correlates with modulation of said EP₁ receptor variant;
c) contacting a second receptor with said compound;
d) determining the level of a corresponding indicator which correlates with modulation of said second receptor; and
e) comparing the level of the indicator from step (b) with the level of the corresponding indicator from step (d), wherein a different level of the indicator from step (b) compared to the level of the corresponding indicator from step (d) indicates that said compound is a compound that differentially modulates said EP₁ receptor variant,
wherein said EP₁ receptor variant is the polypeptide of claim 1.

29. The method of claim 28, wherein said second receptor is a different EP₁ receptor variant.

30. The method of claim 28, wherein said second receptor comprises the amino acid sequence SEQ ID NO: 8, or a functional fragment thereof.

31. The method of claim 28, wherein the level of said indicator from step (b) is greater than the level of said corresponding indicator from step (d).

32. The method of claim 28, wherein the level of said indicator from step (b) is less than the level of said corresponding indicator from step (d).

33. The method of claim 28, wherein said EP₁ receptor variant is a polypeptide comprising an amino acid sequence having at least 80% amino acid identity with SEQ ID NO: 8.

34. The method of claim 28, wherein said EP₁ receptor variant is a polypeptide comprising SEQ ID NO: 2, or a conservative variant thereof.

35. The method of claim 28, wherein said EP₁ receptor variant is an isolated EP₁ receptor polypeptide.

36. The method of claim 28, wherein said EP₁ receptor variant is an EP₁ receptor variant over-expressed in a genetically engineered cell.

37. The method of claim 36, wherein said EP₁ receptor variant is exogenously expressed.

38. The method of claim 28, wherein said indicator in step (b) is calcium.

39. The method of claim 28, wherein said compound is a polypeptide.

40. The method of claim 28, wherein said compound is a small molecule.

41. A method for identifying a compound that differentially binds to an EP₁ receptor variant, comprising:
a) contacting said EP₁ receptor variant with a compound, wherein said EP₁ receptor variant is an isolated EP₁ receptor or an EP₁ receptor variant over-expressed in a genetically engineered cell;
b) determining specific binding of said compound to said EP₁ receptor variant;
c) contacting a second receptor with said compound;
d) determining specific binding of said compound to said second receptor; and
e) comparing the level of specific binding from step (b) with the level of specific binding from step (d), wherein a different level of specific binding from step (b) compared to the level of specific binding from step (d) indicates that said compound is a compound that differentially binds to an EP₁ receptor variant,
wherein said EP₁ receptor variant is the polypeptide of claim 1.

42. The method of claim 41, wherein said second receptor is a different EP₁ receptor variant.

43. The method of claim 41, wherein said second receptor comprises the amino acid sequence SEQ ID NO: 8, or a functional fragment thereof.

44. The method of claim 41, wherein said different level of specific binding is an increased level of binding.

45. The method of claim 41, wherein said different level of specific binding is a decreased level of binding.

46. The method of claim 41, wherein said EP₁ receptor variant is a polypeptide comprising an amino acid sequence having at least 80% amino acid identity with SEQ ID NO: 8.

47. The method of claim 41, wherein said EP₁ receptor variant is a polypeptide comprising SEQ ID NO: 2, or a conservative variant thereof.

48. The method of claim 41, wherein said EP₁ receptor variant is an isolated EP₁ receptor polypeptide.

49. The method of claim 41, wherein said EP₁ receptor variant is an EP₁ receptor variant over-expressed in a genetically engineered cell.

50. The method of claim 49, wherein said EP₁ receptor variant is exogenously expressed.

51. The method of claim 41, wherein said contacting occurs in vitro.

52. The method of claim 41, wherein said compound is a polypeptide.

53. The method of claim 41, wherein said compound is a small molecule.


SEQUENCE LISTING (10 sequences)

SEQ ID NO: 1 - DNA, 1140 bp, Homo sapiens
  CDS (1)...(1131)
atgagcccttgcgggcccctcaacctgagcctggcgggcgaggcgaccacatgcgcggcgccctgggtccccaacacgtcggccgtgccgccgtcgggcgcttcgcccgcgctgcccatcttctccatgacgctgggcgccgtgtccaacctgctggcgctggcgctgctggcgcaggccgcgggccgcctgcgacgccgccgctcggccaccaccttcctgctgttcgtggccagcctgctggccaccgacctggcgggccacgtgatcccgggcgcgctggtgctgcgtctgtacactgcggggcgcgctccggccggcggggcctgccacttcctgggcggctgcatggtcttcttcggcctgtgcccgctgctgctgggctgtggcatggccgtggagcgctgcgtgggcgtcacgcggccgctgctccacgccgcgcgggtctcggtcgcccgcgcgcgcctggcgctggccgcggtggccgcggtggccttggccgtggcgctgctgccgctggcgcgcgtgggccgctatgagctgcagtacccgggcacgtggtgcttcatcggcctgggtcccccgggcggctggcgccaggcactgcttgctggcctcttcgccagcctcggcctggtcgcgctcctcgccgcgctggtgtgcaacacgctcagcggcctggccctgcatcgcgcccgctggcgacgccgctcccgacggcctcccccggcctcaggccccgacagccggcgtcgctggggggcgcacggaccccgctcggcctccgcctcgtccgcctcgtccatcgcttcggcctccaccttctttggcggctctcggagcagcggctcggcacgcagagctcgcgcccacgacgtggagatggtgggccagcttgtcggtatcatggtggtgtcgtgcatctgctggagcccaatgctggtgaggggcgcaccggcccctcgagccacgctccttcccgctccctctcggcaccctcccgccctttgtcgtcccaggacacctggggcctccatcctggactcaaccaaggccccggcccctagaggccccacctgccccgaaagccagcatcgccttctccatctgacctcccatccttcctcctagcccccctct

SEQ ID NO: 2 - protein, 376 aa, Homo sapiens
MSPCGPLNLSLAGEATTCAAPWVPNTSAVPPSGASPALPIFSMTLGAVSNLLALALLAQAAGRLRRRRSATTFLLFVASLLATDLAGHVIPGALVLRLYTAGRAPAGGACHFLGGCMVFFGLCPLLLGCGMAVERCVGVTRPLLHAARVSVARARLALAAVAAVALAVALLPLARVGRYELQYPGTWCFIGLGPPGGWRQALLAGLFASLGLVALLAALVCNTLSGLALHRARWRRRSRRPPPASGPDSRRRWGAHGPRSASASSASSIASASTFFGGSRSSGSARRARAHDVEMVGQLVGIMVVSCICWSPMLVRGAPAPRATLLPAPSRHPPALCRPRTPGASILDSTKAPAPRGPTCPESQHRLLHLTSHPSS

SEQ ID NO: 3 - DNA, 186 bp, Homo sapiens
  CDS (1)...(186)
aggggcgcaccggcccctcgagccacgctccttcccgctccctctcggcaccctcccgccctttgtcgtcccaggacacctggggcctccatcctggactcaaccaaggccccggcccctagaggccccacctgccccgaaagccagcatcgccttctccatctgacctcccatccttcctcctag

SEQ ID NO: 4 - protein, 61 aa, Homo sapiens
RGAPAPRATLLPAPSRHPPALCRPRTPGASILDSTKAPAPRGPTCPESQHRLLHLTSHPSS

SEQ ID NO: 5 - DNA, 30 bp, Homo sapiens
  CDS (1)...(30)
ccaatgctggtgaggggcgcaccggcccct

SEQ ID NO: 6 - protein, 10 aa, Homo sapiens
PMLVRGAPAP

SEQ ID NO: 7 - DNA, 1209 bp, Homo sapiens
  CDS (1)...(1209)
atgagcccttgcgggcccctcaacctgagcctggcgggcgaggcgaccacatgcgcggcgccctgggtccccaacacgtcggccgtgccgccgtcgggcgcttcgcccgcgctgcccatcttctccatgacgctgggcgccgtgtccaacctgctggcgctggcgctgctggcgcaggccgcgggccgcctgcgacgccgccgctcggccgccaccttcctgctgttcgtggccagcctgctggccaccgacctggcgggccacgtgatcccgggcgcgctggtgctgcgtctgtacactgcggggcgcgctccggccggcggggcctgccacttcctgggcggctgcatggtcttcttcggcctgtgcccgctgctgctgggctgtggcatggccgtggagcgctgcgtgggcgtcacgcggccgctgctccacgccgcgcgggtctcggtcgcccgcgcgcgcctggcgctggccgcggtggccgcggtggccttggccgtggcgctgctgccgctggcgcgcgtgggccgctatgagctgcagtacccgggcacgtggtgcttcatcggcctgggtcccccgggcggctggcgccaggcactgcttgctggcctcttcgccagcctcggcctggtcgcgctcctcgccgcgctggtgtgcaacacgctcagcggcctggccctgctacgcgcccgctggcgacgccgctcccgacggcctcccccggcctcaggccccgacagccggcgtcgctggggggcgcacggaccccgctcggcctccgcctcgtccgcctcgtccatcgcttcggcctccaccttctttggcggctctcggagcagcggctcggcacgcagagctcgcgcccacgacgtggagatggtgggccagcttgtcggtatcatggtggtgtcgtgcatctgctggagcccaatgctggtgttggtggcgctggccgtcggcggctggagctctacctccctgcagcggccactattcctggccgtgcgccttgcctcctggaaccagatcctggacccttgggtgtacatcctactgcgccaggccgtgctgcgccaactgcttcgcctcttgcccccgagggccggagccaagggcggccccgcggggctgggcctaacaccgagcgcctgggaggccagctcgctgcgcagctcccggcacagcggcctcagccacttctaa

SEQ ID NO: 8 - protein, 402 aa, Homo sapiens
MSPCGPLNLSLAGEATTCAAPWVPNTSAVPPSGASPALPIFSMTLGAVSNLLALALLAQAAGRLRRRRSAATFLLFVASLLATDLAGHVIPGALVLRLYTAGRAPAGGACHFLGGCMVFFGLCPLLLGCGMAVERCVGVTRPLLHAARVSVARARLALAAVAAVALAVALLPLARVGRYELQYPGTWCFIGLGPPGGWRQALLAGLFASLGLVALLAALVCNTLSGLALLRARWRRRSRRPPPASGPDSRRRWGAHGPRSASASSASSIASASTFFGGSRSSGSARRARAHDVEMVGQLVGIMVVSCICWSPMLVLVALAVGGWSSTSLQRPLFLAVRLASWNQILDPWVYILLRQAVLRQLLRLLPPRAGAKGGPAGLGLTPSAWEASSLRSSRHSGLSHF

SEQ ID NO: 9 - DNA, 20 bp, Homo sapiens
ggtatcatggtggtgtcgtg

SEQ ID NO: 10 - DNA, 19 bp, Homo sapiens
cctggcgcagtaggatgta